(19) United States
(12) Patent Application Publication
(10) Pub. No.: US 2006/0058268 A1
(43) Pub. Date: Mar. 16, 2006

(54) Compounds and methods for treating diabetic vascular diseases

(76) Inventors: Cynthia L. Sundell, Atlanta, GA (US); Charles Kunsch, Norcross, GA (US)

Correspondence Address: Sherry M. Knowles, Esq.;King & Spalding LLP, 191 Peachtree Street, Atlanta, GA, 30303-1763, US

(21) Appl. No.: 11/171,847

(22) Filed: Jun. 30, 2005

Related U.S. Application Data
(60) Provisional application No. 60/584,638, filed on Jul. 1, 2004.

(51) Int. Cl. A61K 31/66; A61K 31/192

(52) U.S. Cl. 514/114; 514/571

(57) ABSTRACT

Compositions and methods of use of compounds of the formula
and pharmaceutically acceptable salts thereof for the treatment of diabetic vascular diseases such as diabetic neuropathy, nephropathy, and retinopathy are described, wherein the substituents of the compound are further defined within the application.

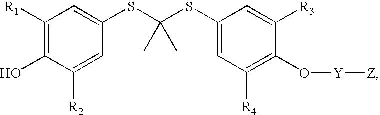

### CROSS REFERENCE TO RELATED APPLICATIONS

This application claims the benefit of U.S. Provisional Application No. 60/584,638, filed Jul. 1, 2004.

### FIELD OF THE INVENTION

The current invention provides compounds, compositions and methods to treat diabetic vascular disease, which includes diabetic retinopathy, diabetic neuropathy, and diabetic nephropathy.

### DESCRIPTION OF RELATED ART

Diabetes, also referred to as diabetes mellitus, is a syndrome characterized by hyperglycemia resulting from absolute or relative impairment in insulin secretion and/or insulin action (The Merck Manual of Diagnosis and Therapy, 17^{th} Ed, Section 2, Chapter 13; Berkow, R., Beers, M. H., and Burs, M., Eds.; John Wiley & Sons, 1999). This disorder is further classified as being either type I diabetes mellitus (DM-1; insulin-dependent DM, IDDM), or type II diabetes mellitus (DM-2), also known as non-insulin-dependent diabetes mellitus. Generally, type I DM is classified as being that type of diabetes most common among those below the age of 30 which is characterized clinically by hyperglycemia and a propensity to develop diabetic ketoacidosis (DKA), wherein the pancreas produces little or no insulin. Type II DM, also characterized by hyperglycemia and insulin resistance, is often associated with visceral/abdominal obesity, has very little or no propensity to ketoacidosis, is typically diagnosed in patients older than 30, and has significant but variable levels of insulin secretion relative to plasma glucose levels.

The primary long-term complication from diabetes is diabetic vascular disease. Diabetic microangiopathy involves the small blood vessels, and gives rise to a variety of lesions depending upon the organ involved. The most reported problems include neuropathy, nephropathy, and retinopathy (Donnelly, R., et al., British Med Journal 320: pp 1062-1066 (2000)).

It has been found that the degree of retinopathy in patients is highly correlated with the duration of the diabetes (Merck Manual, Ch. 99, pp. 729-731). Diabetic retinopathy typically affects those in the age group of 30- to 70-year olds (Aiello, L. P., et al., Diabetes Care 21: pp 143-156 (1998)). Whether or not retinopathy appears depends primarily on the length of time that the patient has had diabetes, and the degree of metabolic control the patient maintains. It has been reported that practically all persons whose diabetes is diagnosed before the age of 30 develop diabetic retinopathy within the first 20 years of diagnosis (Ferris, F. L., et al., New Engl J Med, 341: pp. 667-678 (1999)). Moreover, diabetic retinopathy is already present by the time diabetes is diagnosed in approximately 5% of all the patients age 30 years or older at the time of diagnosis. In the cases of patients who use insulin, this figure rises to 80%. It has been found that only about 20% of those patients who do not require insulin exhibit diabetic retinopathy (Neely, K. A., et al., Med Clin North Am, 82: pp. 847-876 (1998)).

Apart from the high prevalence of these complications of diabetes, their seriousness creates further, more serious problems: 50% of all patients with untreated proliferative retinopathy will lose their sight within five years of the onset of retinopathy (Ferris, F. L., et al., New Engl J Med 341: pp. 667-678 (1999)). Moreover, it has been reported that 3.6% of all patients with DM-1, and 1.6% of all patients with DM-2, are estimated to ultimately go blind (Cunha-Vaz, J. G., Ophthalmologica, 214: pp. 3777-3780 (2000)), although retinopathy is not the sole cause of blindness in all patients with diabetes.

Current treatment approaches to diabetic retinopathy have varied from surgical procedures, such as laser photocoagulation of the retinal lesions, to more chemotherapy-based approaches. It is generally recognized (Arch Ophthalmol, 103: pp. 1796-1806 (1985)) that laser treatment is insufficient, with the retinopathy continuing to progress in a full 50% of the patients who undergo laser photocoagulation. Consequently, as the incidence of diabetic retinopathy increase, so too have the varied approaches to prevention and/or treatment of the disease (Jose Pedro De la Cruz, M.D., et al., Diabetes Metab Res Rev., 20: pp. 91-113 (2004)). Most of the therapeutic approaches have been tailored to one or more of the three stages in the physiopathology of diabetic retinopathy: biochemical alterations caused by hyperglycemia (Stage 1); endothelial dysfunction, including altered anti-thrombotic function, altered regulation of blood flow, and altered mechanisms of control of growth factors (Stage 2); and morphological alterations, such as the failure of retinal vascular function (Stage 3).

Interventions targeted to the first stage of diabetic retinopathy, specifically targeting the biochemical damage caused by prolonged hyperglycemia, is one of the first approaches in the prevention and treatment of this disease. These therapeutic measures can be broken into several approaches; inhibition of the polyol pathway, inhibition of the diacylglycerol-protein kinase C (DAG-PKC) pathway, inhibition of AGEs, and inhibition of oxidative stress with antioxidant drugs.

Aldose reductase (AR) is a key enzyme in polyol formation. In tissues that do not require insulin for cellular glucose uptake (such as the retina and endothelial cells), the glucolytic pathway is overwhelmed in the prolonged hyperglycemia of diabetic retinopathy (Gabbay, K. H., N Engl J Med, 288: pp. 831-836 (1973), Vlassara, H., Diabetes, 46 (suppl. 2): pp. S19-S25 (1997); King, G. L., et al., Endocrinol. Metab Clin North Am, 25: pp. 255-270 (1996)). Therefore, drugs which inhibit polyol pathway flux have been investigated with varied successes (Kato, N., et al., Diabetes Res Clin Pract, 50: pp. 77-85 (2000)). Such drugs include alresatin, sorbinil, tolrestat, epalrestat, and zenalestast. The therapeutic usefulness of these aldose reductase inhibitors has yet to be established (Arch Opthalmol, 108: pp. 1234-1244 (1990)).

Inhibitors of the diacylglycerol-protein kinase C (DAG-PKC) pathway have shown better promise, with both vitamin E (d-alpha-tocopherol; Chappey, O., et al., Eur J Clin Invest, 27: pp. 97-108 (1997)), and LY333 531 (a macrocyclic bisindolylmaleimide compound; Wakasaki, H., et al., Proc Natl Acad Sci, 94: pp. 9320-9325 (1997); Nakamura, J., et al., Diabetes, 48: pp. 2090-2095 (1999)) having a specific inhibitory effect on PKC-β2, the beta isoform of protein kinase C. These results showed increased retinal blood flow in 88% of the patients tested (Bursell, S. E., et al., Diabetes Res Clin Pract, 45: pp. 169-172 (1999)). It was also recently reported (Osicka, T. M., et al., Diabetes, 49: pp. 87-93 (2000)) that the renoprotective substance aminoguanidine reduces renal protein kinase C activity, suggesting its promise for use in the treatment of diabetic retinopathy.

Finally, inhibition of oxidative cellular stress with antioxidant drugs, especially mixtures of antioxidants (e.g., vitamins C and E, β-carotene, and selenium) has shown reductions of up to 65% in the formation of retinal pericyte ghosts (Kowluru, R. A., et al., Diabetes, 50: pp. 1938-1942 (2001); Ceriello, A., et al., Diabetes, 44: pp. 924-928 (1995)). Other substances with potential usefulness include α-lipoic acid (Kern, T. S., et al., Diabetes, 50: pp. 1636-1642 (2001)), aminoguanidine (Morcos, M., et al., Diabetes Res Clin Pract, 52: pp. 175-183 (2001)), as well as Vitamins C and E alone, all of which have shown initial promise as reducing oxidative stress associated with diabetic retinopathy and facilitating the actions of insulin (Paolisso, G., et al., Diabetes Care, 16: pp. 1433-1437 (1993); Reaven, P. D., et al., Diabetes Care, 18: pp. 807-816 (1995)).

The alterations that hyperglycemica causes in the different biochemical pathways, act together in the vascular endothelium to give rise to the second stage of the disease—the so-called endothelial dysfunction. As this dysfunction participates directly in the genesis and progression of diabetic retinopathy, preventative measures aimed at correcting or reducing the effects of endothelial dysfunction are a second significant approach to controlling the disease. Such targeted approaches include the use of platelet function inhibitors such as TxA₂ synthesis inhibitors like aspirin (Diabetes, 38: pp. 491-498 (1989)), trifusal, and ditazol (Esmatjes, E., et al., Diabetes Res Clin Pract, 7: pp. 285-291 (1989); Pagani, A., et al., Curr Ther Res, 45: pp. 409-415 (1989); Moreno, A., et al., Haemostasis, 25: pp166-171 (1995)); increasing the levels of cyclic nucleotides with dipyridamole (Vingolo, E. M., et al., Acta Ophthalmol Scand, 77: pp. 315-329 (1999)), nitrates, and nitrates; and inhibiting the DP pathway with such drugs as ticlodipine (Arch Ophthalmol, 108: 1577-1583 (1990)) and clopidogrel (De la Cruz, J. P., et al. Naunyn-Schmiedeberg's Arch Pharmacol, 367: pp. 204-210 (2002)). Other approaches to the modulation of the endothelium have included non-specific blockage of endothelin receptors (ET_{A} and ET_{B}) with bosentan (Hopfner, R. L., et al., Diabetologia, 42: pp. 1383-1394 (1999)), modulation of prostacyclin synthesis (Shindo, H., et al., Prostaglandins, 41: pp. 85-96 (1991); De La Cruz, J. P., et al., Thromb. Res., 97: pp. 125-131 (2000)), modulation of the nitric oxide pathway by stimulating the production and effects of nitric oxide (NO) with calcium dobesilate (Leite, E. B., et al., Int. Ophthalmol, 14: pp. 81-88 (1990); Ruiz, E., et al., Br. J Pharmacol, 121: pp. 711-716 (1997)), and increasing choroid blood flow with pentoxifylline (Sebag, J., et al., Angiology, 45: pp. 429-433 (1994)). While it has been shown that platelet function inhibitors can slow the progression of diabetic retinopathy, they seem to be effective only if used from the. earliest stages of the disease's progress. Additionally, the high suggested dosage rates of these drugs have led to a high incidence of thrombotic side effects involving their use.

Failure of endothelial function, together with inadequate control of glycemia, leads to the morphological lesions of diabetic retinopathy. These lesions, the third stage of diabetic retinopathy, are often so severe that chemotherapy is no longer an option. In this situation, more aggressive therapeutic measures are often called for. One such intervention is laser coagulation, which can reduce the risk of dramatic visual impairment by inducing the regression of new blood vessels, obliterating infarcted areas, and inducing chorioretinal adhesion (Petrovic, V., et al., Diabetes Technol Ther, 1: pp. 177-187 (1999)). However, the usefulness of this therapeutic approach is often limited to producing little more than a regression of blood vessels and/or a regression of edema by increasing retinal oxygenation (Ooi, C. G., et al., Diabetes Metab Res Rev, 15: pp. 373-377 (1999)). A similar approach, vitrectomy, surgically removes the contents of vitreious hemorrhage and eliminates the medium that favors neovessel growth in proliferative diabetic retinopathy, while simultaneously reducing or minimizing retinal traction (Smiddy, W. E., et al., Surveys of Ophthalmol, 43: pp. 491-507 (1999)). Again, however, the results of using such a surgical approach to date have been less than encouraging (Lewis, H., Am. J. Ophthalmol, 131: pp. 123-125(2001)).

A number of approaches to the treatment of diabetic retinopathy are described in patents as well. U.S. Pat. No. 6,440,933 (issued Aug. 27, 2002) describes peptide derivatives, such as somatostatin analogs, designed to deliver peptides having growth factor inhibitory activity to the retina by sequential metabolism. The peptide derivatives, which reportedly comprise a dihydropyridine pyridinium salt-type redox target or moiety, a bulky lipophilic function and an amino acid/dipeptide/tripeptide spacer, are suggested for use in the prevention and treatment of diabetic retinopathy.

Diabetic neuropathies are a family of nerve disorders caused by diabetes. People with diabetes can, over time, have damage to nerves throughout the body. Neuropathies lead to numbness and sometimes pain and weakness in the hands, arms, feet, and legs. Problems can also occur in every organ system, including the digestive tract, heart, and sex organs. People with diabetes can develop nerve problems at any time, but the longer a person has diabetes, the greater the risk. It has been estimated that 50 percent of those individuals with diabetes have some form of neuropathy, but not all of those with neuropathy have symptoms. Diabetic neuropathy also appears to be more common in people who have had problems controlling their blood glucose levels, in those with high levels of blood fat and blood pressure, in overweight people, and in people over the age of 40. The most common type is peripheral neuropathy, also called distal symmetric neuropathy, which affects the arms and legs.

Symptoms of diabetic neuropathy depend upon the type of neuropathy and which nerves are affected. Some subjects have no symptoms at all. For others, numbness, tingling, or pain in the feet is often the first sign. A person can experience both pain and numbness. Often, symptoms are minor at first, and since most nerve damage occurs over several years, mild cases may go unnoticed for a long time. Symptoms may involve the sensory or motor nervous system, as well as the involuntary (autonomic) nervous system. In some people, mainly those with focal neuropathy, the onset of pain may be sudden and severe.

Diabetic neuropathies can be classified as peripheral, autonomic, proximal, and focal. Each affects different parts of the body in different ways. Peripheral neuropathy causes either pain or loss of feeling in the toes, feet, legs, hands, and arms. Autonomic neuropathy causes changes in digestion, bowel and bladder function, sexual response, and perspiration. It can also affect the nerves that serve the heart and control blood pressure. Autonomic neuropathy can also cause hypoglycemia (low blood sugar) unawareness, a condition in which people no longer experience the warning signs of hypoglycemia. Proximal neuropathy causes pain in the thighs, hips, or buttocks and leads to weakness in the legs. Focal neuropathy results in the sudden weakness of one nerve, or a group of nerves, causing muscle weakness or pain. Any nerve in the body may be affected.

Diabetic nephropathy is a clinical syndrome characterized by persistent albuminuria (>300 mg/d or >200 mcg/min) that is confirmed on at least 2 occasions 3-6 months apart, a relentless decline in the glomerular filtration rate (GFR), and elevated arterial blood pressure. Diabetic nephropathy is the leading cause of chronic renal failure in the United States and other Western societies. It is also one of the most significant long-term complications in terms of morbidity and mortality for individual patients with diabetes.

High blood pressure almost always develops or worsens in diabetic nephropathy, and can be the first abnormality to develop. Diabetic nephropathy is also a sign of worsening blood vessel disease throughout the body. Diabetic eye disease is usually present by this stage indicating damage to smaller blood vessels. Larger blood vessels (arteries) are almost always affected leading to heart attacks, strokes, and circulatory disease occurring more often and at a younger age than usual. Commonly, diabetes will have also resulted in damage to small nerves causing “diabetic peripheral nephropathy” and “autonomic neuropathy”.

U.S. Pat. No. 6,080,732 (issued Jun. 27, 2000) suggests the use of sulodexide, a glycosaminoglycan of natural origin extracted from mammalian intestinal mucosa, and of compositions containing it in the treatment of patients suffering from diabetic retinopathy. The degree of effectiveness of sulodexide is shown by its ability to exhibit improvement of the retinic lesions and by the degree of restoration of the functional integrity of the membrane of the microcapillaries with subsequent decrease of the capillary permeability in diabetic patients treated with pharmaceutical compositions containing the drug.

U.S. Pat. No. 5,639,482 (issued Jun. 17, 1997) describes a method of treating diabetic retinopathy and a means for preventing its reoccurrence by supplementing the diet of diabetics with approximately 1000 mcg sodium selenite and 1000 IU vitamin E on a daily basis for 24 to 35 days until the visual acuity of the diabetic patient improves. The patent also suggests that following the vitamin treatment regimen, a daily maintenance supplement of 250 mcg sodium selenite and 400 IU vitamin E can be continued.

U.S. Pat. No. 5,019,591 (issued May 28, 1991) describes a method for treating and preventing retinopathy and for treating and preventing other small vessel complications associated with diabetes which comprises administering an antihistamine or a pharmaceutically acceptable derivative of an antihistamine to a patient having retinopathy. In one embodiment of the patent, the antihistamine is selected from the group of diphenhydramine, terfenadine, mequitazine, astemizole, acrivastine, SCH 29851, SK&F 93944, clemastine, ketotifen, azatadine, oxatomide, azelastine, doxepine, piperoxan (933F), 929F, 1571F, mepyramine, chlorpheniramine, triprolidine and promethazine, while in a second embodiment, the antihistamine is described as being burimamide, cimetidine, ranitidine, famotidine or nizatidine.

U.S. Pat. No. 5,262,439 to Parthasarathy, which is assigned to AtheroGenics, Inc. discloses analogs of probucol with increased water solubility in which one or both of the hydroxyl groups are replaced with ester groups that increase the water solubility of the compound. In one embodiment, the ester is formed from dicarboxylic acids selected from the group consisting of a mono- or di-probucol ester of succinic acid, glutaric acid, adipic acid, seberic acid, sebacic acid, azelaic acid, or maleic acid. In another embodiment, the probucol derivative is a mono- or di-ester in which the ester contains an alkyl or alkenyl group that contains a functionality selected from the group consisting of a carboxylic acid group, amine group, salt of an amine group, amide groups, amide groups, and aldehyde groups.

U.S. Pat. No. 5,155,250 to Parker, et al. discloses that 2,6-dialkyl-4-silylphenols are antiatherosclerotic agents. The same compounds are disclosed as serum cholesterol lowering agents in PCT Publication No. WO 95/15760, published on Jun. 15, 1995. U.S. Pat. No. 5,608,095 to Parker, et al. discloses that alkylated-4-silyl-phenols inhibit the peroxidation of LDL, lower plasma cholesterol, and inhibit the expression of VCAM-1, and thus are useful in the treatment of atherosclerosis.

U.S. Pat. No. 6,121,319, which issued on Sep. 19, 2000, and corresponding WO 98/51662 filed by AtheroGenics, Inc. and published on Nov. 18, 1998, discloses certain compounds of formula having the formula 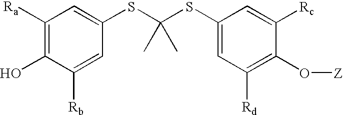
wherein:

Rₐ, R_{b}, R_{c}, and R_{d} are independently any group that does not otherwise adversely affect the desired properties of the molecule, including hydrogen, straight chained, branched, or cyclic alkyl which may be substituted, aryl, substituted aryl, heteroaryl, substituted heteroaryl, alkaryl, substituted alkaryl, aralkyl or substituted aralkyl; substituents on the Rₐ, R_{b}, R_{c} and R_{d} groups are selected from the group consisting of hydrogen, halogen, alkyl, nitro, amino, haloalkyl, alkylamino, dialkylamino, acyl, and acyloxy; Z is selected from the group consisting of hydrogen, alkyl, substituted alkyl, alkenyl, substituted alkenyl, alkynyl, substituted alkynyl, aryl, aralkyl, alkaryl, heteroaryl, heteroaralkyl, a carbohydrate group, —(CH₂)—Rₑ, —C(O)—R_{g}, and —C(O)—(CH₂)ₙ—Rₕ, wherein (a) when each of Rₐ, R_{b}, R_{c}, and R_{d} are t-butyl, Z cannot be hydrogen; and the other variables are as defined in those specifications, for the treatment of disorders mediated by VCAM-1, and inflammatory and cardiovascular disorders.

WO 01/70757 filed by AtheroGenics, Inc. and published on Sep. 27, 2001, describes the use of certain thioethers of the following formula (I), and pharmaceutically acceptable salts thereof: 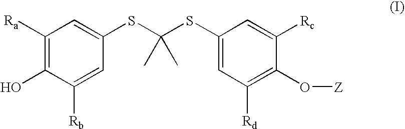
wherein

Rₐ, R_{b}, R_{c}, and R_{d} are independently any group that does not adversely affect the desired properties of the molecule, including hydrogen, alkyl, substituted alkyl, aryl, substituted aryl, heteroaryl, substituted heteroaryl, alkaryl, substituted alkaryl, aralkyl, or substituted aralkyl; and Z is (i) a substituted or unsubstituted carbohydrate, (ii) a substituted or unsubstituted alditol, (iii) C₁₋₁₀ alkyl or substituted C₁₋₁₀ alkyl, terminated by sulfonic acid, (iv) C₁₋₁₀ alkyl or substituted C₁₋₁₀ alkyl, terminated by phosphonic acid, (v) substituted or unsubstituted C₁₋₁₀ alkyl-O—C(O)—C₁₋₁₀ alkyl, (vi) straight chained polyhydroxylated C₃₋₁₀ alkyl; (vii) —(CR₂)₁₋₆—COOH, wherein R is independently hydrogen, halo, amino, or hydroxy, and wherein at least one of the R substituents is not hydrogen; or (viii) —(CR₂)₁₋₆—X, wherein X is aryl, heteroaryl, or heterocycle, and R is independently hydrogen, halo, amino, or hydroxy.

U.S. Pat. No. 6,147,250, issued to AtheroGenics, Inc. on Nov. 14, 2000, provides a compound, composition and method for inhibiting the expression of VCAM-1, and thus can be used in the treatment of a disease mediated by VCAM-1, which includes administering a compound of (II), or a pharmaceutically acceptable salt thereof, optionally in a pharmaceutically acceptable carrier. The compounds of formula (II) are: 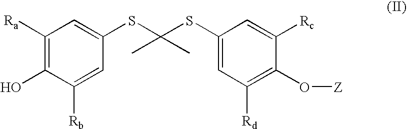
wherein

Rₐ, R_{b}, R_{c}, and R_{d} are independently any group that does not otherwise adversely affect the desired properties of the molecule, including hydrogen, straight chained, branched, or cyclic alkyl which may be substituted, aryl, substituted aryl, heteroaryl, substituted heteroaryl, alkaryl, substituted alkaryl, aralkyl or substituted aralkyl; substituents on the Rₐ, R_{b}, R_{c} and R_{d} groups are selected from the group consisting of hydrogen, halogen, alkyl, nitro, amino, haloalkyl, alkylamino, dialkylamino, acyl, and acyloxy; Z is selected from the group consisting of hydrogen, alkyl, substituted alkyl, alkenyl, substituted alkenyl, alkynyl, substituted alkynyl, aryl, aralkyl, alkaryl, heteroaryl, heteroaralkyl, carbohydrate group, —(CH₂)—Rₑ, —C(O)—R_{g}, and —C(O)—(CH₂)ₙ —Rₕ, wherein (a) when each of Rₐ, R_{b}, R_{c}, and R_{d} are t-butyl, Z cannot be hydrogen; Rₑ is selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR, NR₂, mono- or polyhydroxy-substituted alkyl, aryl, substituted aryl, heteroaryl, substituted heteroaryl, acyloxy, substituted acyloxy, COOH, COOR, —CH(OH)Rₖ, hydroxy, C(O)NH₂, C(O)NHR, C(O)NR₂; R_{g} is selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR, NR₂, mono- or polyhydroxy-substituted alkyl, aryl, substituted aryl, heteroaryl, substituted heteroaryl; and Rₕ is selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR, NR₂, mono- or polyhydroxy-substituted alkyl, aryl, substituted aryl, heteroaryl, substituted heteroaryl, acyloxy, substituted acyloxy, COOH, COOR, —CH(OH)Rₖ, hydroxy, O-phosphate, C(O)NH₂, C(O)NHR, C(O)NR₂ and pharmaceutically acceptable salts thereof.

Meng, et al. discloses a series of phenolic compounds that has been discovered as potent inhibitors of TNF-α-inducible expression of vascular cell adhesion molecule-I (VCAM-1) with concurrent antioxidant and lipid-modulating properties. The compounds disclosed have demonstrated efficacies in animal models of atherosclerosis and hyperlipidemia (Bioorganic & Med Chem Ltrs., 12(18): 2545-2548 ( 2002)).

Similarly, Sundell et al. described a novel metabolically stable phenolic antioxidant compound derived from probucol, ([4-[[1-[[3,5-bis(1,1-dimethy- lethyl)-4-hydroxypehenyl]thio]-1-methylethyl]thio]2,6-bis(1,1-dimethylethyl)phenoxy]acetic acid), that inhibits TNF-α-stimulated endothelial expression of VCAM-1 and MCP-1, two redox-sensitive inflammatory genes critical for the recruitment of leukocytes to joints in rheumatoid arthritis (RA), to a greater extent than ICAM-1 (FASEB Journal, 16: p. A182 (2002); Apr. 20-24, 2002, Annual Meeting of the Professional Research Scientists on Experimental Biology, ISSN 0892-6638).

Given the range of strong side effects associated with the current proposed therapeutic regimens that are used in treating diabetic vascular disease, including retinopathy, neuropathy, and nephropathy, as well as the limited successes of these regimens in treating the underlying conditions of this pathologic disorder to date, there exists a need for new methods and compositions useful in the treatment of diabetic vascular disease, as well as other small vessel complications which arise in connection with diabetes.

It is an object of the present invention to provide new compositions, methods, and strategies for use in treating diabetic vascular diseases, including diabetic retinopathy, diabetic neuropathy, and diabetic nephropathy, as well as disorders associated with the progression of such diabetic vascular diseases.

It is another object of the present invention to provide new compositions, methods, and strategies that simultaneously exhibit strong activity against diabetic vascular diseases in a patient and exhibit a minimal effect on normal cells in the patient.

It is a further object of the present invention to provide new compositions, methods, and strategies that simultaneously exhibit strong activity against the progression of diabetic vascular disease, such as diabetic retinopathy, neuropathy, and nephropathy, in a patient and simultaneously exhibit a minimal effect on normal cells within the patient.

It is yet a further object of the present invention to provide compositions, methods, and strategies for the treatment of a patient having a diabetic vascular disease such as diabetic retinopathy, neuropathy, and nephropathy, which are sufficiently stable to be stored until use in an appropriate composition and administered by any suitable and desired mode.

In a further object of the present invention, compositions, methods, and strategies for the treatment of a patient having an ocular inflammatory disorder which may or may not be associated with diabetes, such as uveitis, retinal vasculitis, or other inflammatory diseases of the conjunctiva, cornea, sclera, retina and orbit are provided, as well as disorders associated with the progression of such ocular inflammatory diseases.

### SUMMARY OF THE INVENTION

The present invention provides, in one embodiment, a method of inhibiting or treating diabetic vascular disease in a host (typically a mammal, and more typically a human) in need thereof, including retinopathy, neuropathy, and nephropathy, wherein the method comprises administering, either alone or in combination with other medications, a therapeutically effective amount of a compound of Formula I 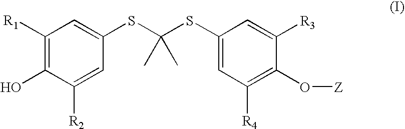
or a pharmaceutically acceptable salt thereof, wherein:

R₁, R₂, R₃, and R₄ are independently hydrogen, straight chained, branched (for example, tert-butyl), or cyclic alkyl which may be substituted, aryl, substituted aryl heteroaryl, substituted heteroaryl, alkaryl, substituted alkaryl, aralkyl or substituted aralkyl; substituents on the R₁, R₂, R₃ and R₄ groups are selected from the group consisting of hydrogen, halogen, alkyl, nitro, amino, haloalkyl, alkylamino, dialkylamino, acyl, and acyloxy;

Z is selected from the group consisting of hydrogen, alkyl, substituted alkyl, alkenyl, substituted alkenyl, alkynyl, substituted alkynyl, aryl, aralkyl, alkaryl, heteroaryl, heteroaralkyl, carbohydrate group, —(CH₂)ₙ—R₅, —C(O)—R₆, and —C(O)—(CH₂)ₙ —R₇, wherein (a) when each of R₁, R₂, R₃, and R₄ are t-butyl, Z cannot be hydrogen and, (b) n is0, 1, 2, 3, 4, 5, 6, 7, 8, 9,or 10;

R₅ is selected from the group consisting of alkyl, substituted alkyl, alkenyl, substituted alkenyl, alkynyl, substituted alkynyl, alkoxy, substituted alkoxy, alkoxyalkyl, substituted alkoxyalkyl, NH₂, NHR, NR₂, mono- or polyhydroxy-substituted alkyl, aryl, substituted aryl, heteroaryl, substituted heteroaryl, acyloxy, substituted acyloxy, COOH, COOR, —CH(OH)Rₖ, hydroxy, C(O)NH₂, C(O)NHR, C(O)NR₂;

R₆ is selected from the group consisting of alkyl, substituted alkyl, alkenyl, substituted alkenyl, alkynyl, substituted alkynyl, alkoxy, substituted alkoxy, alkoxyalkyl, substituted alkoxyalkyl, NH₂, NHR, NR₂, mono- or polyhydroxy-substituted alkyl, aryl, substituted aryl, heteroaryl, and substituted heteroaryl;

R₇ is selected from the group consisting of alkyl, substituted alkyl, alkenyl, substituted alkenyl, alkynyl, substituted alkynyl, alkoxy, substituted alkoxy, alkoxyalkyl, substituted alkoxyalkyl, NH₂, NHR, NR₂, mono- or polyhydroxy-substituted alkyl, aryl, substituted aryl, heteroaryl, substituted heteroaryl, acyloxy, substituted acyloxy, COOH, COOR, —CH(OH)R₈, hydroxy, O-phosphate, C(O)NH₂, C(O)NHR, C(O)NR₂ and pharmaceutically acceptable salts thereof.

Rₖ and R₈ are independently selected from the group consisting of H, alkyl, substituted alkyl, alkenyl, substituted alkenyl, alkynyl, substituted alkynyl, alkoxy, substituted alkoxy, alkoxyalkyl, substituted alkoxyalkyl, NH₂, NHR, NR₂, mono- or polyhydroxy-substituted alkyl, aryl, substituted aryl, heteroaryl, and substituted heteroaryl;

R is independently selected from the group consisting of H, alkyl, substituted alkyl, alkenyl, substituted alkenyl, alkynyl, substituted alkynyl, alkoxy, substituted alkoxy, alkoxyalkyl, substituted alkoxyalkyl, mono- or polyhydroxy-substituted alkyl, aryl, substituted aryl, heteroaryl, and substituted heteroaryl;

In an alternative embodiment, R₅, R₆, and R₇ can independently be a substituent which improves the water solubility of the compound, including, but not limited to C(O)-spacer-SO₃H, wherein spacer is as defined below, C(O)-spacer-SO₃M, wherein M is a metal used to form a pharmaceutically acceptable salt, for example, sodium, C(O)-spacer-PO₃H₂, C(O)-spacer-PO₃M₂, C(O)-spacer-PO₃HM, C(O)-spacer-PO₄H, C(O)-spacer-PO₄M, SO₃M, —PO₃H₂, —PO₃M₂, —PO₃HM, cyclic phosphates, polyhydroxyalkyl, carbohydrate groups, C(O)-spacer-[O(C₁₋₃ alkyl)ₚ]ₙ, wherein n is as defined above and p is 1, 2, or 3, —[O(C₁₋₃ alkyl)ₚ]ₙ, carboxy lower alkyl, lower alkylcarbonyl lower alkyl, N,N-dialkyl amino lower alkyl, pyridyl lower alkyl, imidazolyl lower alkyl, morpholinyl lower alkyl, pyrrolidinyl lower alkyl, thiazolinyl lower alkyl, piperidinyl lower alkyl, morpholinyl lower hydroxyalkyl, N-pyrryl, piperazinyl lower alkyl, N-alkyl piperazinyl lower alkyl, triazolyl lower alkyl, tetrazolyl lower alkyl, tetrazolylamino lower alkyl, or thiazolyl lower alkyl.

Spacer, as used herein, is a group selected from the group consisting of —(CH₂)ₙ—, —(CH₂)ₙ—CO—, —(CH₂)ₙ—N—, —(CH₂)ₙ—O—, —(CH₂)ₙ—S—, —(CH₂O)—, —(OCH₂)—, —(SCH₂)—, —(CH₂S—), -(aryl-O)—, —(O-aryl)-, -(alkyl-O)—, or —(O-alkyl)-.

Substituents on the groups defined above are selected from the group consisting of alkyl, alkenyl, alkynyl, hydroxy, halo, nitro, amino, alkylamino, dialkylamino, carboxy, aryl, heteroaryl, COOR, CONH₂, CONHR, CONR₂, haloalkyl, alkoxyalkyl, mono- or polyhydroxyalkyl, CH₂OR, CH₂OH, OCOR, O-phosphate, SO₂NH₂, SO₂NHR, or SO₂NR₂.

In another embodiment of the present invention, a method of inhibiting or treating diabetic vascular disease, including retinopathy, neuropathy, and nephropathy, in a host in need thereof (typically a mammal, and more typically a human) is provided, wherein the method comprising administering an effective amount of a compound of Formula (II) 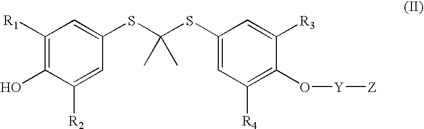
or a pharmaceutically acceptable salt thereof, wherein:

Y is a bond or

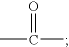

R₁, R₂, R₃ and R₄ are independently selected from the group consisting of hydrogen, hydroxy, C₁₋₁₀ alkyl, aryl, heteroaryl, C₋₁₀ alkaryl, and aryl C₁₋₁₀ alkyl, wherein all nonhydrogen and hydroxy substituents may optionally be substituted from one or more of the group selected from C₁₋₁₀ alkyl, halogen, nitro, amino, halo C₁₋₁₀ alkyl, C₁₋₁₀ alkylamino, di C₁₋₁₀ alkylamino, acyl, and acyloxy;

Z is selected from the group consisting of C₁₋₁₀ alkyl, C₂₋₁₀ alkenyl, C₂₋₁₀ alkynyl, hydroxyl C₁₋₁₀ alkyl, aryl, heteroaryl, C₁₋₁₀ alkaryl, aryl C₁₋₁₀ alkyl, heteroaryl C₁₋₁₀ alkyl, C₁₋₁₀ alkoxy C₁₋₁₀ alkyl, C₁₋₁₀ alkylamino C₁₋₁₀ alkyl, carboxy C₁₋₁₀ alkyl, C₁₋₁₀ dialkylamino C₁₋₁₀ alkyl, amino C₁₋₁₀ alkyl, heterocycle, heterocycl C₁₋₁₀ alkyl, R₇NH, R₇ R₇N, carboxy, carbohydrate group, carbohydrate lactone group, and an alditol group wherein all may optionally be substituted by one or more R₅;

R₅ is independently selected from the group selected from the group selected from hydroxy, C₁₋₁₀ alkyl, C₁₋₁₀ alkoxy, halo, nitro, amino, cyano, C₁₋₁₀ alkylamino, diC₁₋₁₀ alkylamino, acyl, acyloxy, COOH, COOR₇, OC(O)R₇, CH(OH)R₇, NHR₇, NR₇ R₇, C(O)NH₂, C(O)NHR₇, CONR₇R₇, NHC(O)OR₇, OSO₃H, SO₃H, SO₂ NHR₇, SO₂ NR₇ R₇, P(O)(OH)OR₇, PO₂H₂P(O)(OH)R₇, P(O)(OR₇)₂, P(O)R₇(OR₇), OPO₃H, PO₃H, PO₃H₂, hydroxymethyl, and cyclic phosphate, wherein when possible, all may be optionally substituted by one or more R₆;

R₆ is independently selected from the group consisting of hydroxy, C₁₋₁₀ alkyl, C₁₋₁₀ alkoxy, acyloxy, halo, nitro, amino, cyano, haloC₁₋₁₀ alkyl, C₁₋₁₀ alkylamino, diC₁₋₁₀ alkylamino, acyl, and acyloxy;

R₇ is independently selected from the group consisting of C₁₋₁₀ alkyl, C₂₋₁₀ alkenyl, C₂₋₁₀ alkynyl, C₁₋₁₀ alkoxy, C₁₋₁₀ alkoxycarbonyl C₁₋₁₀ alkyl, aryl, carboxy C₁₋₁₀ alkyl, C₁₋₁₀ alkylcarboxy C₁₋₁₀ alkyl, C₁₋₁₀ alkylcarboxy C₁₋₁₀ aryl, heterocycle, heterocycl C₁₋₁₀ alkyl, and heteroaryl, wherein all may be optionally substituted by one or more R₈ ; and

R₈ is independently selected from the group consisting of hydroxy, C₁₋₁₀ alkyl, C₁₋₁₀ alkoxy, acyloxy, halo, nitro, amino, cyano, and carboxy; wherein two R₇ groups may come together to form a 4 to 7 membered ring.

In a further embodiment of the present invention, a method of inhibiting or treating diabetic vascular disease, including retinopathy, neuropathy, and nephropathy, in a host in need thereof is described, wherein the method comprises administering, either alone or in combination with other medications, a therapeutically effective amount of a compound of Formula III 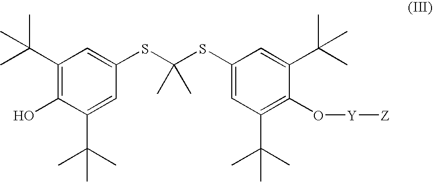
or a pharmaceutically acceptable salt thereof, wherein:

Y is a bond or

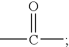

Z is selected from the group consisting of C₁₋₁₀ alkyl, C₂₋₁₀ alkenyl, C₂₋₁₀ alkynyl, hydroxyl C₁₋₁₀ alkyl, aryl, heteroaryl, C₁₋₁₀ alkaryl, aryl C₁₋₁₀ alkyl, heteroaryl C₁₋₁₀ alkyl, C₁₋₁₀ alkoxy C₁₋₁₀ alkyl, C₁₋₁₀ alkylamino C₁₋₁₀ alkyl, carboxy C₁₋₁₀ alkyl, C₁₋₁₀ dialkylamino C₁₋₁₀ alkyl, amino C₁₋₁₀ alkyl, heterocycle, heterocycl C₁₋₁₀ alkyl, R₇NH, R₇R₇N, carboxy, carbohydrate group, carbohydrate lactone group, and an alditol group wherein all may optionally be substituted by one or more R₅;

R₅ is independently selected from the group selected from the group selected from hydroxy, C₁₋₁₀ alkyl, C₁₋₁₀ alkoxy, halo, nitro, amino, cyano, C₁₋₁₀ alkylamino, diC₁₋₁₀ alkylamino, acyl, acyloxy, COOH, COOR₇, OC(O)R₇, CH(OH)R₇, NHR₇, NR₇ R₇, C(O)NH₂, C(O)NHR₇, CONR₇ R₇, NHC(O)OR₇, OSO₃H, SO₃H, SO₂NHR₇, SO₂NR₇R₇, P(O)(OH)OR₇, PO₂H₂P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;

R₆ is independently selected from the group consisting of hydroxy, C₁₋₁₀ alkyl, C₁₋₁₀ alkoxy, acyloxy, halo, nitro, amino, cyano, haloC₁₋₁₀ alkyl, C₁₋₁₀ alkylamino, diC₁₋₁₀ alkylamino, acyl, and acyloxy;

R₇ is independently selected from the group consisting of C₁₋₁₀ alkyl, C₂₋₁₀ alkenyl, C₂₋₁₀ alkynyl, C₁₋₁₀ alkoxy, C₁₋₁₀ alkoxycarbonyl C₁₋₁₀ alkyl, aryl, carboxy C₁₋₁₀ alkyl, C₁₋₁₀ alkylcarboxy C₁₋₁₀ alkyl, C₁₋₁₀ alkylcarboxy C₁₋₁₀ aryl, heterocycle, heterocycl C₁₋₁₀ alkyl, and heteroaryl, wherein all may be optionally substituted by one or more R₈ ; and

R₈ is independently selected from the group consisting of hydroxy, C₁₋₁₀ alkyl, C₁₋₁₀ alkoxy, acyloxy, halo, nitro, amino, cyano, and carboxy; wherein two R₇ groups may come together to form a 4 to 7 membered ring.

In yet another embodiment of the present disclosure, a method of inhibiting or treating diabetic vascular disease, including retinopathy, neuropathy, and nephropathy in patients is described, wherein the method comprises administering, either alone or in combination with other medications, a compound selected from the group of consisting of 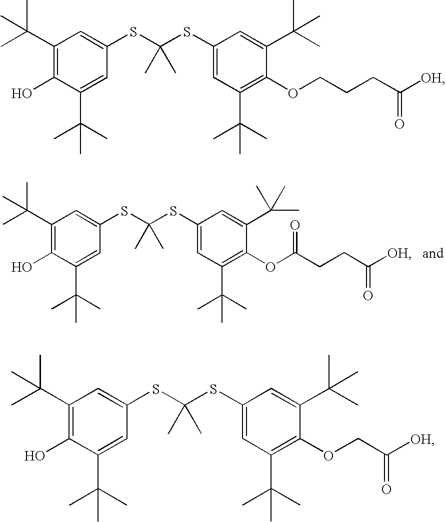
or pharmaceutically acceptable salts thereof.

In a further embodiment of the present disclosure, a method of inhibiting or treating diabetic vascular disease, including retinopathy, neuropathy, and nephropathy, in patients, either alone or in combination with other medications, is described, wherein the method comprises administering a therapeutically effective amount of a compound of formula 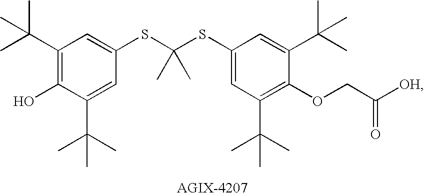
or a pharmaceutically acceptable salt thereof.

In a further embodiment of the present invention, a pharmaceutical composition for the treatment and/or prophylaxis of ocular inflammatory disorders, which may or may not be associated with diabetes, such as uveitis, retinal vasculitis, or other inflammatory diseases of the conjunctiva, cornea, sclera, retina, and orbit, is described, the composition comprising a compound of Formula I, Formula II, or Formula III as disclosed herein in any of the previous embodiments, or a pharmaceutically acceptable salt or prodrug thereof, optionally with a pharmaceutically acceptable carrier or diluent, and optionally with one or more other effective therapeutic agents.

In another embodiment of the present invention, a pharmaceutical composition for the treatment and/or prophylaxis of a diabetic vascular disease in a mammal, especially a mammal diagnosed as having or being at risk for such a disease, is described, the composition comprising a compound of Formula I, Formula II, or Formula III as disclosed herein in any of the previous embodiments, or a pharmaceutically acceptable salt or prodrug thereof, optionally with a pharmaceutically acceptable carrier or diluent, and optionally with one or more other effective therapeutic agents for the treatment of vascular disorders.

As a further embodiment of the present invention, a method for the treatment of a diabetic vascular disease in a mammal is described, comprising administering an effective amount of a compound of Formula I, Formula II or Formula III as disclosed in previous embodiments, or a pharmaceutically acceptable salt or prodrug thereof, optionally with a pharmaceutically acceptable carrier, excipient or diluent, and optionally in combination and/or alternation with one or more other effective therapeutic agents for the treatment of vascular disorders.

In another embodiment of the present invention, the use of a compound of Formula I, Formula II, or Formula III as disclosed in previous embodiments, or a pharmaceutically acceptable salt or prodrug thereof, optionally with a pharmaceutically acceptable carrier or diluent, for the treatment of a diabetic vascular disease in a mammal, optionally in combination and/or alternation with one or more other effective therapeutic agents, is described.

In yet a further embodiment of the present invention, the use of a compound of Formula I, Formula II or Formula III as disclosed in previous embodiments, or a pharmaceutically acceptable salt or prodrug thereof, optionally in combination and/or alternation with one or more other effective therapeutic agents, and optionally with a pharmaceutically acceptable carrier or diluent, in the manufacture of a medicament for the treatment of a diabetic vascular disease in a mammal is described.

### DESCRIPTION OF THE FIGURES

FIG. 1 shows inhibition of basal intracellular ROS levels upon treatment with compounds of the present disclosure.

FIG. 2 shows shows the measurements of leukostasis upon treatment of retinas with compounds of the present invention.

FIG. 3 shows the measurements of hemodynamics upon treatment of retinas with compounds of the present invention.

FIG. 4 shows a summary of treatment effects based on the study of VCAM-1 mRNA levels.

### DETAILED DESCRIPTION OF THE INVENTION

A method of treating diabetic vascular diseases, especially diabetic neuropathy, diabetic nephropathy, and diabetic retinopathy, in patients diagnosed with such disease is provided. In accordance with the present invention, retinal blood flow may be increased, vasoconstrictor overexpression may be decreased, and/or retinal microvascular abnormalities may be prevented. Combinations of drugs comprising the compounds of the present disclosure in treatment regimens for diabetic vascular diseases and/or ocular inflammatory diseases are also contemplated herein.

The present invention envisions methods for the treatment of diabetic vascular diseases, including diabetic neuropathy, nephropathy, and retinopathy in patients, including stage 1, stage 2, and stage 3 diabetic retinopathy. The method for treatment of a patient currently afflicted with a diabetic vascular disease, in accordance with the present disclosure, comprises administering to the patient in need of such treatment a therapeutically effective amount of a compound of Formula I or Formula II, or Formula III, 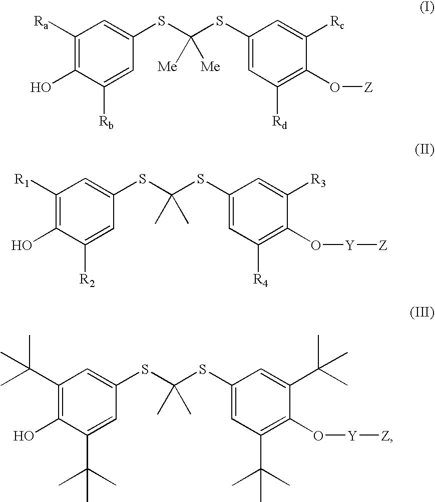
or a pharmaceutically acceptable salt thereof, wherein the variable designations (R₁, R₂, R₃, R_{4,} R₅, Y and Z) are as defined previously above. The therapeutically effective amount administered is preferably in the form of a pharmaceutical formulation comprising the compound and a suitable carrier or excipient therefore. Such a pharmaceutical formulation, in accordance with the present invention, can also include flavors, binders, lubricants, inert diluents, lubricating, surface active or dispersing agents, and numerous other additives known in the art of pharmaceutical formulations.
I. Definitions.

The terms “C₁-C₁₀ alkyl”, “C₂-C₁₀ alkenyl”, C₁-C₁₀ alkoxy, C₂-C₁₀ alkenoxy, C₂-C₁₀ alkynyl, and C₂-C₁₀ alkynoxy are considered to include, independently, each member of the group, such that, for example, C₁-C₁₀ alkyl includes straight, branched and where appropriate cyclic C₁, C₂, C₃, C₄, C₅, C₆, C₇, C₈, C₉ and C₁₀ alkyl functionalities; C₂-C₁₀ alkenyl includes straight, branched, and where appropriate cyclic C₂, C₃, C₄, C₅, C₆, C₇, C₈, C₉ and C₁₀ alkenyl functionalities; C₁-C₁₀ alkoxy includes straight, branched, and where appropriate cyclic C₁, C₂, C₃, C₄, C₅, C₆, C₇, C₈, C₉ and C₁₀ alkoxy functionalities; C₂-C₁₀ alkenoxy includes straight, branched, and where appropriate cyclic C₂, C₃, C₄, C₅, C₆, C₇, C₈, C₉ and C₁₀ alkenoxy functionalities; C₂-C₁₀ alkynyl includes straight, branched and where appropriate cyclic C₁, C₂, C₃, C₄, C₅, C₆, C₇, C₈, C₉ and C₁₀ alkynyl functionalities; and C₂-C₁₀ alkynoxy includes straight, branched, and where appropriate cyclic C₂, C₃, C₄, C₅, C₆, C₇, C₈, C₉ and C₁₀ alkynoxy functionalities.

The term “alkyl”, alone or in combination, means an acyclic, saturated straight, branched, or cyclic, primary, secondary, or tertiary hydrocarbon, including those containing from 1 to 10 carbon atoms or from 1 to 6 carbon atoms. Said alkyl radicals may be optionally substituted with groups as defined below. The term alkyl specifically includes but is not limited to methyl, ethyl, propyl, isopropyl, cyclopropyl, butyl, isobutyl, t-butyl, sec-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl, heptyl, octyl; nonyl, decyl, trifluoromethyl and difluoromethyl. The term includes both substituted and unsubstituted alkyl groups. Moieties with which the alkyl group can be substituted are, for example, alkyl, hydroxyl, halo, nitro, cyano, alkenyl, alkynyl, heteroaryl, heterocyclic, carbocycle, alkoxy, oxo, aryloxy, arylalkoxy, cycloalkyl, tetrazolyl, heteroaryloxy; heteroarylalkoxy, carbohydrate, amino acid, amino acid esters, amino acid amides, alditol, haloalkylthi, haloalkoxy, haloalkyl, hydroxyl, carboxyl, acyl, acyloxy, amino, aminoalkyl, aminoacyl, amido, alkylamino, dialkylamino, arylamino, nitro, cyano, thiol, imide, sulfonic acid, sulfate, sulfonate, sulfonyl, alkylsulfonyl, aminosulfonyl, alkylsulfonylamino, haloalkylsulfonyl, sulfanyl, sulfinyl, sulfamoyl, carboxylic ester, carboxylic acid, amide, phosphonyl, phosphinyl, phosphoryl, thioester, thioether, oxime, hydrazine, carbamate, phosphonic acid, phosphate, phosphonate, phosphinate, sulfonamido, carboxamido, hydroxamic acid, sulfonylimide or any other desired functional group that does not inhibit the pharmacological activity of this compound, either unprotected, or protected as necessary, as known to those skilled in the art, for example, as taught in Greene, et al., Protective Groups in Organic Synthesis, John Wiley and Sons, Third Edition, 1999, hereby incorporated by reference.

The term “alkenyl”, alone or in combination, means an acyclic, straight, branched, or cyclic, primary, secondary, or tertiary hydrocarbon, including those containing from 2 to 10 carbon atoms or from 2 to 6 carbon atoms, wherein the substituent contains at least one carbon-carbon double bond. Said alkenyl radicals may be optionally substituted. Examples of such radicals include but are not limited to are ethylene, methylethylene, and isopropylidene.

The term “alkynyl” refers to an unsaturated, acyclic hydrocarbon radical, linear or branched, in so much as it contains one or more triple bonds, including such radicals containing about 2 to 10 carbon atoms or having from 2 to 6 carbon atoms. The alkynyl radicals may be optionally substituted with groups as defined herein. Examples of suitable alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl radicals and the like.

The term “acyl”, alone or in combination, means a carbonyl or thionocarbonyl group bonded to any desired radical including but not limited to, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” are formyl, acetyl, benzoyl, trifluoroacetyl, phthaloyl, malonyl, nicotinyl, and the like.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having one or more alkoxy radicals attached to the alkyl radical, that is, to form monoalkoxyalkyl and dialkoxyalkyl radicals. Other alkoxy radicals are “lower alkoxy” radicals having one to six carbon atoms. Examples of such radicals include methoxy, ethoxy, propoxy, butoxy and tert-butoxy alkyls. The “alkoxy” radicals may be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “alkoxy” is defined as —OR, wherein R is alkyl, including cycloalkyl, as defined above.

The term “alkoxyalkyl” is defined as an alkyl group wherein a hydrogen has been replaced by an alkoxy group. The term “(alkylthio)alkyl” is defined similarly as alkoxyalkyl, except a sulfur atom, rather than an oxygen atom, is present.

The term “alkylthio” and “arylthio” are defined as —SR, wherein R is alkyl or aryl, respectively.

The term “alkylsulfinyl” is defined as R—SO₂, wherein R is alkyl.

The term “alkylsulfonyl” is defined as R—SO₃, wherein R is alkyl.

The term “aryl”, alone or in combination, means a carbocyclic aromatic system containing one, two or three rings wherein such rings may be attached together in a pendent manner or may be fused. Examples of aryl groups include phenyl, benzyl and biphenyl. The “aryl” group can be optionally substituted where possible with one or more of the desired moieties including but not limited to alkyl, hydroxyl, halo, nitro, cyano, alkenyl, alkynyl, heteroaryl, heterocyclic, carbocycle, alkoxy, oxo, aryloxy, arylalkoxy, cycloalkyl, tetrazolyl, heteroaryloxy; heteroarylalkoxy, carbohydrate, amino acid, amino acid esters, amino acid amides, alditol, haloalkylthi, haloalkoxy, haloalkyl, hydroxyl, carboxyl, acyl, acyloxy, amino, aminoalkyl, aminoacyl, amido, alkylamino, dialkylamino, arylamino, nitro, cyano, thiol, imide, sulfonic acid, sulfate, sulfonate, sulfonyl, alkylsulfonyl, aminosulfonyl, alkylsulfonylamino, haloalkylsulfonyl, sulfanyl, sulfinyl, sulfamoyl, carboxylic ester, carboxylic acid, amide, phosphonyl, phosphinyl, phosphoryl, thioester, thioether, oxime, hydrazine, carbamate, phosphonic acid, phosphate, phosphonate, phosphinate, sulfonamido, carboxamido, hydroxamic acid, sulfonylimide or any other desired functional group that does not inhibit the pharmacological activity of this compound, either unprotected, or protected as necessary, as known to those skilled in the art. In addition, adjacent groups on an “aryl” ring may combine to form a 5- to 7-membered saturated or partially unsaturated carbocyclic, aryl, heteroaryl or heterocyclic ring, which in turn may be substituted as above.

The term “carboxyalkyl” refers to a carboxy group attached to an alkyl group.

The term “halo” as defined herein refers to fluoro, bromo, chloro, and iodo.

The term “hydroxyalkyl” refers to radicals wherein any one or more of the alkyl carbon atoms is substituted with a hydroxyl (—OH) functionality. Nonlimiting examples of hydroxyalkyl include monohydroxyalkyl, dihydroxyalkyl, and polyhydroxyalkyl radicals.

The term “heterocyclic” refers to a nonaromatic cyclic group that may be partially (contains at least one double bond) or fully saturated and wherein there is at least one heteroatom, such as oxygen, sulfur, nitrogen, or phosphorus in the ring. The term heteroaryl or heteroaromatic, as used herein, refers to an aromatic that includes at least one sulfur, oxygen, nitrogen or phosphorus in the aromatic ring. Nonlimiting examples of heterocylics and heteroaromatics are pyrrolidinyl, tetrahydrofuryl, piperazinyl, piperidinyl, morpholino, thiomorpholino, tetrahydropyranyl, imidazolyl, pyrolinyl, pyrazolinyl, indolinyl, dioxolanyl, or 1,4-dioxanyl. aziridinyl, furyl, furanyl, pyridyl, pyrimidinyl, benzoxazolyl, 1,2,4-oxadiazolyl, 1,3,4-oxadiazolyl, 1,3,4-thiadiazole, indazolyl, 1,3,5-triazinyl, thienyl, isothiazolyl, imidazolyl, tetrazolyl, pyrazinyl, benzofuranyl, quinolyl, isoquinolyl, benzothienyl, isobenzofuryl, pyrazolyl, indolyl, isoindolyl, benzimidazolyl, purinyl, carbazolyl, oxazolyl, thiazolyl, benzothiazolyl, isothiazolyl, 1,2,4-thiadiazolyl, isooxazolyl, pyrrolyl, quinazolinyl, cinnolinyl, phthalazinyl, xanthinyl, hypoxanthinyl, pyrazole, imidazole, 1,2,3-triazole, 1,2,4-triazole, 1,2,3-oxadiazole, thiazine, pyridazine, or pteridinyl wherein said heteroaryl or heterocyclic group can be optionally substituted with one or more substituent selected from the same substituents as set out above for aryl groups. Functional oxygen and nitrogen groups on the heteroaryl group can be protected as necessary or desired. Suitable protecting groups can include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl.

The term “therapeutically effective amount” or “therapeutically effective dose” shall mean that amount of drug or pharmaceutical agent that will elicit the biological or medical response of a tissue, system, animal or human that is being sought. The term “therapeutically effective dose” refers to that amount of the compound which results in achieving the desired effect. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio of LD₅₀ to ED50. Compounds that exhibit high therapeutic indices (i.e., a toxic dose that is substantially higher than the effective dose) are preferred. The data obtained can be used in formulating a dosage range for use in humans. The dosage of such compounds preferably lies within a range of circulating concentrations that include the ED₅₀ with little or no toxicity. The dosage can vary within this range depending upon the dosage form employed, and the route of administration utilized.

The term “diabetic vascular disease”, as used herein, refers to those diseases and disorders linked to diabetes which effect either large or small blood vessels. Included in this classification are peripheral vascular diseases as well as neuropathy, nephropathy (kidney failure), and retinopathy.

The term “diabetic retinopathy”, as used herein, refers to those ocular disorders wherein diabetes or the complications of diabetes damages blood vessels in the retina of the eye, and includes both non-proliferative diabetic retinopathy (NPDR) and proliferative diabetic retinopathy (PDR).

The term “host”, as used herein, refers to a cell or organism that exhibits the properties associated with diabetic vascular disease or ocular inflammatory diseases. The hosts are typically vertebrates, including both birds and mammals, and are more preferably mammals. It is preferred that the mammal, as a host or patient in the present disclosure, is from the family of Primates, Carnivora, Proboscidea, Perissodactyla, Artiodactyla, Rodentia, and Lagomorpha. It is even more preferable that the mammal vertebrate of the present invention be Canis familiaris (dog), Felis catus (cat), Elephas maximus (elephant), Equus caballus (horse), Sus domesticus (pig), Camelus dromedarious (camel), Cervus axis (deer), Giraffa camelopardalis (giraffe), Bos taurus (cattle/cows), Capra hircus (goat), Ovis aries (sheep), Mus musculus (mouse), Lepus brachyurus (rabbit), Mesocricetus auratus (hamster), Cavia porcellus (guinea pig), Meriones unguiculatus (gerbil), and Homo sapiens (human). Most preferably, the host or patient as used within the present invention is Homo sapiens (human). Birds suitable as hosts within the confines of the present invention include Gallus domesticus (chicken) and Meleagris gallopavo (turkey).

II. Formulations and Administration

1. Formulation

Hosts, including mammals and particularly humans, suffering from any of the disorders described herein, including both diabetic vascular disorders and ocular inflammatory disorders, can be treated by administering to the host an effective amount of a compound of Formula I, Formula II, or Formula III as described herein, or a pharmaceutically acceptable prodrug, ester, and/or salt thereof, optionally in combination with a pharmaceutically acceptable carrier or diluent. The active materials can be administered by any appropriate route, for example, orally, parenterally, intravenously, intradermally, intramuscularly, subcutaneously, sublingually, transdermally, bronchially, pharyngolaryngeal, intranasally, topically such as by a cream or ointment, rectally, intraarticular, intracisternally, intrathecally, intravaginally, intraperitoneally, intraocularly, by inhalation, bucally or as an oral or nasal spray.

The compounds of the present invention can be used in the form of pharmaceutically acceptable salts derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zürich, Switzerland: 2002). The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the present invention include phosphate, tris and acetate.

Pharmaceutically acceptable salts may be also obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids can also be made.

The formulations may conveniently be presented in unit dosage form and may be prepared by any of the methods well known in the art of pharmacy. All methods include the step of bringing into association a compound of the invention or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

The compound or a pharmaceutically acceptable ester, salt, solvate or prodrug can be mixed with other active materials that do not impair the desired action, or with materials that supplement the desired action, including other drugs against diabetic vascular disease or ocular inflammatory disease. Solutions or suspensions used for parenteral, intradermal, subcutaneous, or topical application can include, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants including immunostimulating factors (including immunostimulatory nucleic acid sequences, including those with CpG sequences), preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

Besides inert diluents, the formulation compositions can also include adjuvants such as wetting agents, emulsifying and suspending agents, sweetening, flavoring, and perfuming agents.

The active compounds can also be in micro-or nano-encapsulated form, if appropriate, with one or more excipients.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Formulations for parenteral (including subcutaneous, intradermal, intramuscular, intravenous and intraarticular) administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

The method of the invention may be practiced using pharmaceutical formulations containing compounds of Formula I, Formula II, or Formula III. Treatment of diabetic vascular disease or ocular inflammatory disorders in a human may be therapeutic by administering compound of Formula I, Formula II, or Formula III to treat an existing condition so as to mitigate the effects of that event. Alternatively, and equally acceptable, treatment of diabetic vascular disease or ocular inflammatory disorders in a human may be prophylactic by administering a compound of Formula I, Formula II, or Formula III in anticipation of a worsening condition of diabetic vascular disease, for example, in a patient whose occupation, lifestyle, or exposure to irritants will expectedly worsen an existing condition of the diabetic vascular disease or ocular inflammatory disorder. In some cases, the underlying cause of the disease state will not be prevented or cured, but may be reduced in severity or extent and its symptoms ameliorated by administration of compounds of Formula I, Formula II, or Formula III (and their formulations) using the method of the invention.

2. Administration

The compounds of the invention are administered by any appropriate administration route, for example, orally, parenterally, intravenously, intradermally, intramuscularly, subcutaneously, sublingually, transdermally, bronchially, pharyngolaryngeal, intranasally, topically such as by a cream or ointment, rectally, intraarticular, intracisternally, intrathecally, intravaginally, intraperitoneally, intraocularly, by inhalation, bucally or as an oral or nasal spray. The route of administration may vary, however, depending upon the condition and the severity of the diabetic vascular disease or ocular inflammation. The precise amount of compound administered to a host or patient will be the responsibility of the attendant physician. However, the dose employed will depend on a number of factors, including the age and sex of the patient, the precise disorder being treated, and its severity. In accordance with the compositions of the present invention, a dose range of from about 0.001 mg/kg per day to about 2500 mg/kg per day is typical. For example, the dose range is from about 0.1 mg/kg per day to about 1000 mg/kg per day. Optionally, the dose range is from about 0.1 mg/kg per day to about 500 mg/kg per day, including 1 mg/kg, 2 mg/kg, 5 mg/kg, 10 mg/kg, 15 mg/kg, 20 mg, kg, 25 mg/kg, 30 mg/kg, 35 mg/kg, 40 mg/kg, 45 mg/kg, 50 mg/kg, 100 mg/kg, 200 mg/kg, 300 mg/kg, 400 mg/kg, 500 mg/kg per day, and values between any two of the values given in this range. The dose range for humans is for example from about 0.005 mg to 100 g/day. Alternatively, the dose range in accordance with the present invention is such that the blood serum level of compounds of the present invention is from about 0.01 μM to about 100 μM, and preferably from about 0.1 μM to about 100 μM. Suitable values of blood serum levels in accordance with the present invention include but are not limited to about 0.01 μM, about 0.1 μM, about 0.5 μM, about 1 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM and about 100 μM, as well as any blood serum level that falls within any two of these values (e.g, between about 10 μM and about 60 μM). Tablets or other forms of dosage presentation provided in discrete units may conveniently contain an amount of one or more of the compounds of the invention which are effective at such dosage rages, or ranges in between these ranges.

3. Combinations

The compounds of the present invention may also be administered alone or as part of a composition allowing for a controlled release of the therapeutically active compound. In general, the compound of Formula I, Formula II, or Formula III, or pharmaceutically acceptable salts thereof, will be administered to a mammal such as man so that a therapeutically effective amount is received. A therapeutically effective amount may conventionally be determined for an individual patient by administering the active compound in increasing doses and observing the effect on the patient, for example, reduction of symptoms associated with the particular diabetic vascular condition. Generally, the compound must be administered in a manner and a dose to achieve in the human the desired blood level concentration of a compound of Formula I, Formula II, or Formula III needed to exhibit a therapeutic effect.

Compounds of the present invention may be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, excipients, and the like. The preferred lipids are the natural and synthetic phospholipids and phosphatidylcholines (lecithins) used separately or together. Methods to form liposomes are known in the art. See, for example, Prescott, Ed., Methods in Cell Biology, Volume XIV, Academic Press, New York, N.Y., (1976), p 33 et seq. and U.S. Pat. No. 4,522,811. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

The compounds of the present invention can be administered alone, in a pharmaceutical composition, as a pharmaceutically acceptable salt, or in combination or alteration with one or more therapeutic drugs, including any used in connection with diabetic vascular disorders such as diabetic retinopathy, diabetic neuropathy, and diabetic nephropathy. Particularly included are compounds mentioned in the Background of the Invention, or in Table A. For example, compounds of the present invention may be administered with one or more drugs selected from the group consisting of corticosteroids such as dexamethasone and fluocindone acetonide, cyclosporine, calcium channel agonists, tobramycin, protein Kinase C beta inhibitors (PKC-β inhibitors), anti-vascular endothelial growth factors (anti-VEGF), aspirin, dipyridamole, clopidogrel, meloxicam and eternacept, as well as their derivatives.

TABLE A
Examples of Diabetic Vascular Disorder Drugs

| Generic Name | Brand Name/Development Name |
|---|---|
| Aspirin | numerous |
| Vitamin E | |
| Dexamethasone | Decadron ® |
| Fluocindone acetonide | Fluonide ® |
| Dipyridamole | Persantine ® |
| Clopidogrel | Plavix ® |
| Meloxicam | Mobic ® |
| Trifusal | |
| Ticlodipine | Ticlid ® |
| Calcium dobesilate | Dobesil ® |
| Pentoxifylline | Trental |
| Bosentan | Tracleer ® |
| anti-VEGF | RhuFab (Genentech) |
| Eternacept | ENBREL ® |
| PKC-β-inhibitor | LY333531 (Eli Lilly) |

4. Dosage Forms

The compounds and formulations of the present invention can be administered in any of the known dosage forms standard in the art; in solid dosage form, semi-solid dosage form, or liquid dosage form, as well as subcategories of each of these forms.

Solid dosage forms for oral administration include capsules, caplets, tablets, pills, powders, lozenges, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Compositions for rectal or vaginal administration are for example suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Semil-liquid dosage forms include those dosage forms that are too soft in structure to qualify for solids, but to thick to be counted as liquids. These include creams, pastes, ointments, gels, lotions, and other semisolid emulsions containing the active compound of the present invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Dosage forms for topical or transdermal administration of a compound of this invention include ointments, pastes, creams, lotions, gels, powders, solutions, sprays, inhalants or patches, optionally mixed with degradable or nondegradable polymers. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

Formulations containing compounds of the invention may be administered through the skin by an appliance such as a transdermal patch. Patches can be made of a matrix such as polyacrylamide, polysiloxanes, or both and a semi-permeable membrane made from a suitable polymer to control the rate at which the material is delivered to the skin. Other suitable transdermal patch formulations and configurations are described in U.S. Pat. Nos. 5,296,222 and 5,271,940, as well as in Satas, D., et al, “Handbook of Pressure Sensitive Adhesive Technology, 2^{nd} Ed.”, Van Nostrand Reinhold, 1989: Chapter 25, pp. 627-642.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

III. Controlled-Release Formulations

In one embodiment, the active compounds of the present invention are prepared with carriers that will protect the compound against rapid elimination from the body or rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylacetic acid. Methods for preparation of such formulations will be apparent to those skilled in the art.

The field of biodegradable polymers has developed rapidly since the synthesis and biodegradability of polylactic acid was reported by Kulkarni, et al. (“Polylactic acid for surgical implants,” Arch. Surg, 1966, 93, 839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), U.S. Pat. No. 4,767,628 to Hutchinson (polylactide, polylactide-co-glycolide acid), and U.S. Pat. No. 4,530,840 to Tice, et al. (polylactide, polyglycolide, and copolymers). See also U.S. Pat. No. 5,626,863 to Hubbell, et al which describes photopolymerizable biodegradable hydrogels as tissue contacting materials and controlled release carriers (hydrogels of polymerized and crosslinked macromers comprising hydrophilic oligomers having biodegradable monomeric or oligomeric extensions, which are end capped monomers or oligomers capable of polymerization and crosslinking); and PCT WO 97/05185 filed by Focal, Inc. directed to multiblock biodegradable hydrogels for use as controlled release agents for drug delivery and tissue treatment agents.

Degradable materials of biological origin are well known, for example, crosslinked gelatin. Hyaluronic acid has been crosslinked and used as a degradable swelling polymer for biomedical applications (U.S. Pat. No. 4,957,744 to Della Valle et. al.; “Surface modification of polymeric biomaterials for reduced thrombogenicity,” Polym. Mater. Sci. Eng., 1991, 62, 731-735]).

Many dispersion systems are currently in use as, or being explored for use as, carriers of substances, particularly biologically active compounds. Dispersion systems used for pharmaceutical and cosmetic formulations can be categorized as either suspensions or emulsions. Suspensions are defined as solid particles ranging in size from a few manometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442 issued to Haynes. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 to Dunn, et al., discloses a method for forming an implant in situ by dissolving a non-reactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

U.S. Pat. No. 5,718,921 discloses microspheres comprising polymer and drug dispersed there within. U.S. Pat. No. 5,629,009 discloses a delivery system for the controlled release of bioactive factors. U.S. Pat. No. 5,578,325 discloses nanoparticles and microparticles of non-linear hydrophilic hydrophobic multiblock copolymers. U.S. Pat. No. 5,545,409 discloses a delivery system for the controlled release of bioactive factors. U.S. Pat. No. 5,494,682 discloses ionically cross-linked polymeric microcapsules.

U.S. Pat. No. 5,728,402 to Andrx Pharmaceuticals, Inc. describes a controlled release formulation that includes an internal phase which comprises the active drug, its salt, ester or prodrug, in admixture with a hydrogel forming agent, and an external phase which comprises a coating which resists dissolution in the stomach. U.S. Pat. Nos. 5,736,159 and 5,558,879 to Andrx Pharmaceuticals, Inc. discloses a controlled release formulation for drugs with little water solubility in which a passageway is formed in situ. U.S. Pat. No. 5,567,441 to Andrx Pharmaceuticals, Inc. discloses a once-a-day controlled release formulation. U.S. Pat. No. 5,508,040 discloses a multiparticulate pulsatile drug delivery system. U.S. Pat. No. 5,472,708 discloses a pulsatile particle based drug delivery system. U.S. Pat. No. 5,458,888 describes a controlled release tablet formulation which can be made using a blend having an internal drug containing phase and an external phase which comprises a polyethylene glycol polymer which has a weight average molecular weight of from 3,000 to 10,000. U.S. Pat. No. 5,419,917 discloses methods for the modification of the rate of release of a drug to form a hydrogel which is based on the use of an effective amount of a pharmaceutically acceptable ionizable compound that is capable of providing a substantially zero-order release rate of drug from the hydrogel. U.S. Pat. No. 5,458,888 discloses a controlled release tablet formulation.

U.S. Pat. No. 5,641,745 to Elan Corporation, plc discloses a controlled release pharmaceutical formulation which comprises the active drug in a biodegradable polymer to form microspheres or nanospheres. The biodegradable polymer is suitably poly-D,L-lactide or a blend of poly-D,L-lactide and poly-D,L-lactide-co-glycolide. U.S. Pat. No. 5,616,345 to Elan Corporation plc describes a controlled absorption formulation for once a day administration that includes the active compound in association with an organic acid, and a multi-layer membrane surrounding the core and containing a major proportion of a pharmaceutically acceptable film-forming, water insoluble synthetic polymer and a minor proportion of a pharmaceutically acceptable film-forming water soluble synthetic polymer. U.S. Pat. No. 5,641,515 discloses a controlled release formulation based on biodegradable nanoparticles. U.S. Pat. No. 5,637,320 discloses a controlled absorption formulation for once a day administration. U.S. Pat. Nos. 5,580,580 and 5,540,938 are directed to formulations and their use in the treatment of neurological diseases. U.S. Pat. No. 5,533,995 is directed to a passive transdermal device with controlled drug delivery. U.S. Pat. No. 5,505,962 describes a controlled release pharmaceutical formulation.

IV. General Synthetic Methods

Methods for the preparation of the compounds of the present invention are disclosed, for example, in U.S. Pat. Nos. 6,147,250 and 6,670,398, among others, both of which are incorporated herein by reference.

It is appreciated that compounds of the present invention having a chiral center may exist in and be isolated in optically active and racemic forms. Some compounds may also exhibit polymorphism. It is to be understood that the present invention encompasses any racemic, optically-active, diastereomeric, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following:

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme;

iv) enzymatic asymmetric synthesis—a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

Some of the compounds of the present invention can exist in tautomeric, geometric or stereoisomeric forms. The present invention contemplates all such compounds, including cis- and trans-geometric isomers, E- and Z-geometric isomers, R- and S-enantiomers, diastereomers, D-isomers, L-isomers, the racemic mixtures thereof and other mixtures thereof, as falling within the scope of the invention. Pharmaceutically acceptable salts of such tautomeric, geometric or stereoisomeric are also included within the invention. The terms “cis” and “trans” denote a form of geometric isomerism in which two carbon atoms connected by a double bond will each have two high ranking groups on the same side of the double bond (“cis”) or on opposite sides of the double bond (“trans”). Some of the compounds described herein contain alkenyl groups, and are meant to include both cis and trans, or “E” and “Z”, geometric forms. Some of the compounds described contain one or more stereocenters and are meant to include R, S, and mixtures of R and S forms for each stereocenter present.

Some of the compounds described herein may contain one or more ketonic or aldehydic carbonyl groups or combinations thereof alone or as part of a heterocyclic ring system. Such carbonyl groups may exist in part or principally in the “keto” form and in part or principally as one or more “enol” forms of each aldehyde and ketone group present. Compounds of the present invention having aldehydic or ketonic carbonyl groups are meant to include both “keto” and “enol” tautomeric forms.

Some of the compounds described herein may contain one or more imine or enamine groups or combinations thereof. Such groups may exist in part or principally in the “imine” form and in part or principally as one or more “enamine” forms of each group present. Compounds of the present invention having said imine or enamine groups are meant to include both “imine” and “enamine” tautomeric forms.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Measurement of Intracellular ROS

Bovine retinal endothelial cells (BRECs) [VEC Technologies, Inc., Rensselaer, N.Y.; or, Dr. David Antonetti, Pennsylvania State University] were cultivated on fibronectin-coated cell culture flasks with MCDB-131 complete medium (VEC Technologies, Inc., Rensselaer, N.Y.). Pre-confluent cells were treated with AGIX-4207 compound (either at 5 μM or 10 μM) for 1 hour, followed by co-treatment with TNF-α (10 ng/mL) for 2 hours. Cells were then lysed and the total RNA samples isolated by RNeasy reagent (Quiagen, Inc., Hilden, Germany). Real-time PCR assay was performed with primer pairs specific for VCAM-1 or GAPDH. Values represent an average of two independent experiments.

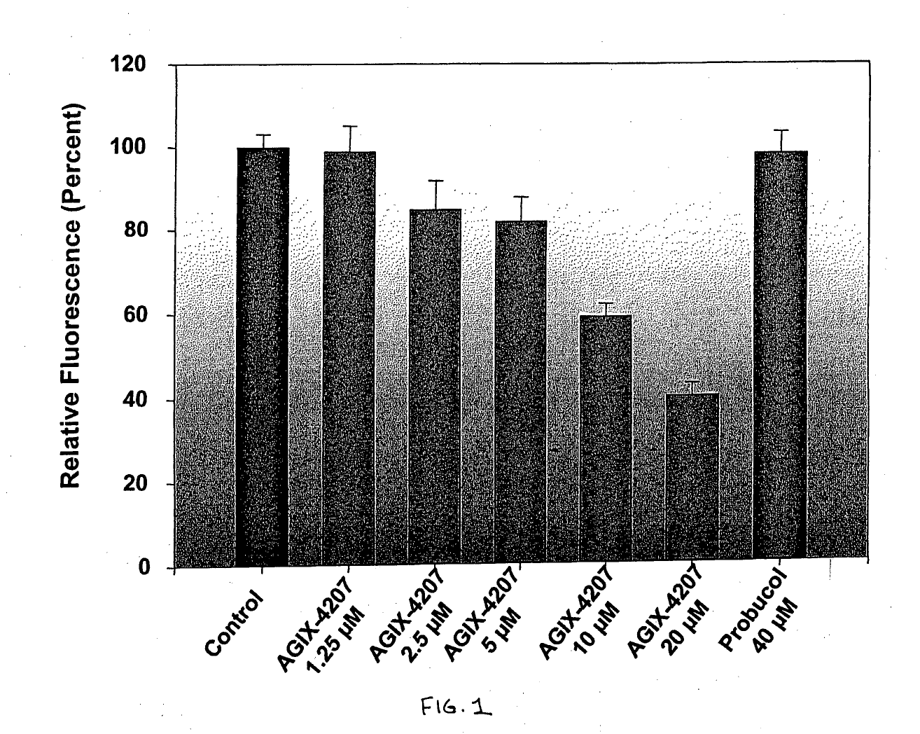

The basal intracellular reactive oxygen species (ROS) were measured using the intracellular oxidation of 2′,7′-dichlorodihydrofluorescein-diacetate (H₂DCF-DA; Molecular Probes, Eugene, Oreg.). In the intracellular compartment, esterases cleave off the acetate group on H₂DCF-DA, resulting in the entrapment of H₂DCF in the intracellular compartment. Upon exposure to oxidants, intracellular H₂DCF is oxidized into a fluorescent compound (DCF) that can be measured by an increase in fluorescence intensity at 530 nm when the sample is excited at 485 nm. Bovine retinal endothelial cells (BRECS) were pretreated with various concentrations of AGIX-4207 for 3 hours, followed by co-treatment with 10 mM H₂DCF-Da for 30 minutes. The cells were then washed with PBS, lysed in Tris-Buffered Saline (TBS)-Tween, and the DCF fluorescence measured. The results, shown in FIG. 1, are reported as percent fluorescence relative to the untreated sample.

### Example 2

### Measurement of Leukostasis

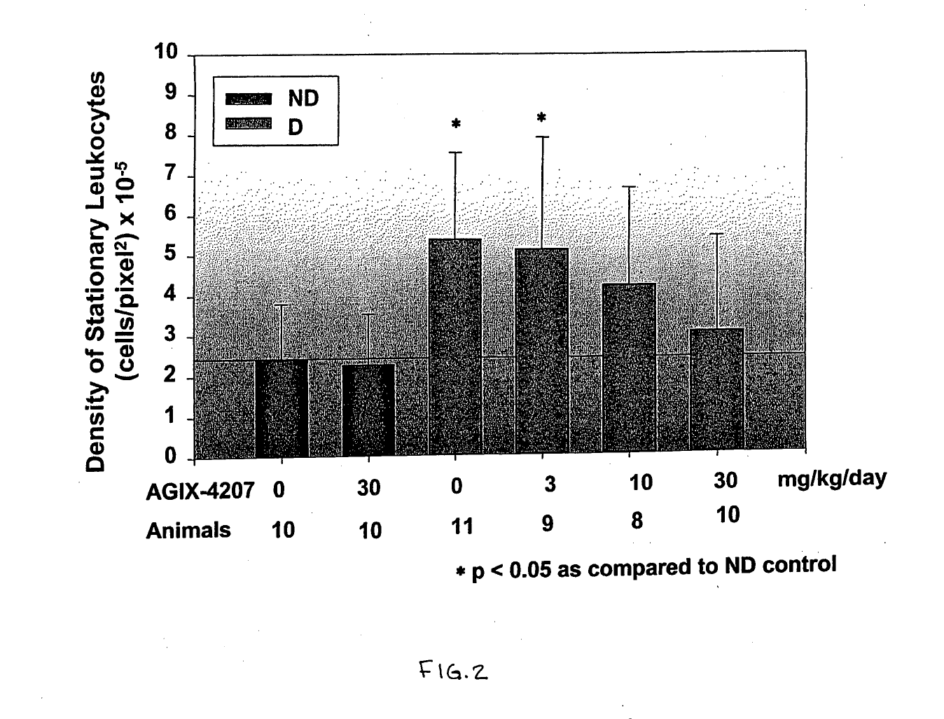

Retinal leukostasis of the compounds of the present disclosure were measured following the techniques as described by Miyamoto (Miyamoto, K., et al., Proc. Natl. Acad. Sci., USA, 96 (19): pp. 10836-10841 (1999)). Acridine orange (Sigma, Milwaukee, Wis.), 4 mg/kg, was infused through a jugular catheter at a rate of approximately 1.5 mL/min. After twenty minutes, the fundus was imaged by SLO to quantify static leukocytes in the retina. Leukostatis, shown in FIG. 2, is represented by total leukocyte number/area (pixels²) between large vessels. An average of 9 quadrants was taken, which represents an area up to 3 disk diameters.

### Example 3

### Measurement of Retinal Hemodynamics

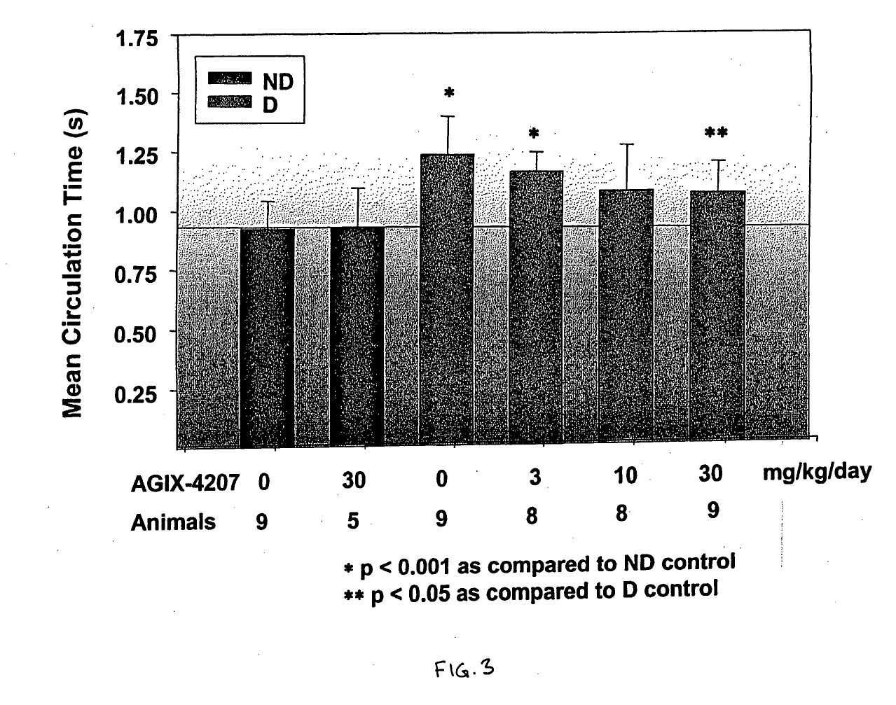

Retinal parameters were assessed by video fluorescein angiography (VFA) using a scanning laser opthalmoscope (SLO) and digital capture of the video output as described in the art (Clermont A. C., et al., Invest Ophthalmol Via Sci, 35: pp.981-990, (1994)). Video angiograms were recorded contralaterally, using a 40° field and 5 μl bolus of 10% sodium fluorescein. Dye dilution curves were analyzed using the techniques described by Bursell. Mean circulation time, MCT, was reported as the difference between the mean filling time of the veins and the arteries, and is inversely proportional to the retinal blood flow. This data is shown in FIG. 3.

### Example 4

### Measurement of Real-Time PCR.

Rat retinal tissues were dissected from streptozotozin (STZ)-induced diabetic rats, and stored in Trizol reagent. Retinal tissue was homogenized, and total RNA extracted and purified using the Trizol method (Life Technologies; Chomczynski, P., et al., Anal. Biochem., 162: pp. 156-159 (1987)). cDNA was generated using an iScript cDNA Synthesis Kit (Bio-Rad Laboratories). The initial amounts of pro-inflammatory gene transcripts were quantitatively assessed by real-time PCR using the SYBR Green Method with a cDNA template, gene specific primers (e.g., VCAM-1 or GAPDH), and the core mix reagent (Qiagen or Bio-Rad Laboratories). The level of each specific mRNA expression was normalized by the corresponding level of GAPDH mRNA.

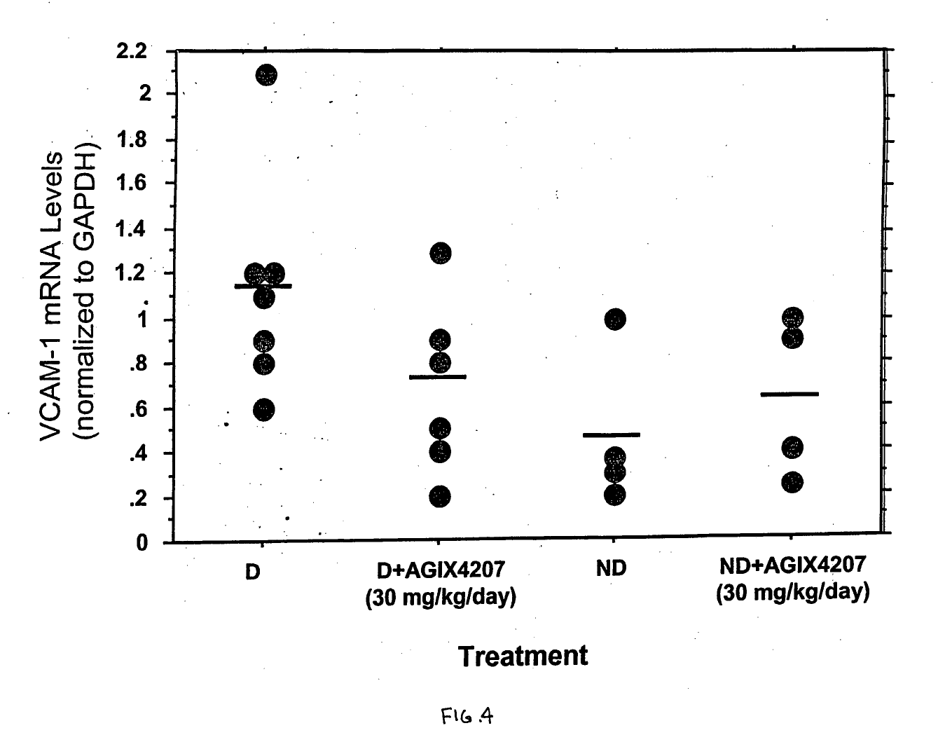

These in vitro test results were expressed statistically as mean ±SEM. Statistical comparisons were made with the Student's t-test method after the analysis of variance (ANOVA) by StarView statistical analysis software program. All in vivo results are expressed as mean ±SD. Multiple comparisons were made using Students-Newman-Keuls tests for normal distributions for the Kruskal-Wallis one-way analysis on ranks using Dunn's method for unequal variance. Results were considered significantly different at P<0.05. FIG. 4 illustrates the results of these measurements.

As is apparent from the data associated with the above experiments, compounds of the present invention reduced reactive oxygen species and VCAM-1 mRNA expression in TNF-α stimulated bovine retinal endothelial cells. Further, the studies of the present invention illustrate that orally administered compounds of the present invention normalized both MCT and RBF by at least 50%, normalized leukostasis by 87%, normalized retinal vascular permeability by at least 69%, and normalized retinal VCAM-1 mRNA levels in diabetic patients by at least 70%.

### Example 5

### VCAM-1 Assay

Splitting the Cells:

Two to four confluent P150 plates are trypsinized and the cells transferred to a 50 mL conical centrifuge tube. The cells are pelleted, resuspended, and counted using the trypan blue exclusion method.

Cells are resuspended at a concentration of 36,000 cells/mL and 1 mL is aliquoted per well.

Cells are split into 24 well tissue culture plates. The cells in each well should be approximately 90-95% confluent by the following day. Cells should not be older than passage 8.

Preparation of Compounds:

Water Soluble Compounds

Compounds are initially screened at 50 μM and 10 μM. A 50 mM stock solution for each compound is prepared in culture medium. The stock solution is diluted to 5 mM and 1 mM. When 10 μL of the 5 mM solution is added to the well (1 mL medium/well), the final concentration will be 50 μM. Adding 10 μL of the 1 mM solution to the well will give a final concentration of 10 μM.

Poorly Soluble Compounds

Compounds which will not go into solution in culture medium are resuspended in DMSO at a concentration of 25 mM. The stock solution is then diluted to the final concentration in culture medium. The old medium is aspirated and 1 mL of the new medium with the compound is added. For example, if the final concentration is 50 .mu.M, the 2 μL of the 25 mM stock is added per mL of culture medium. The 50 mM solution is diluted for lower concentrations.

Adding the Compounds

The compounds are added to the plate (each compound is done in duplicate). One plate is done for VCAM expression and one plate is done for ICAM expression.

Immediately after the compounds are added, TNF is added to each well. 100 units/mL TNF is usually added to each well. Since each lot of TNF varies in the number of units, each new lot is titrated to determine the optimum concentration. Therefore this concentration will change. If 100 units/mL is being used, dilute the TNF to 10 units/μL and add 10 μL to each well.

The plates are incubated at 37° C., 5% CO₂ overnight (approximately 16 hours). The next day the plates are checked under the microscope to see if there are any visual signs of toxicity. Records are made of any cell death, debris, or morphology changes, as well as insoluble compounds (particulate or turbity).

### Example 6

### ELISA Assay

In order to assess MCP-1, the media (500 μL) is saved and frozen at −70° C. Wash cells once with roughly 1 ml/well of Hanks Balance Salt Solution (HBSS) or PBS. Gently empty the wash solution and then tap the plate onto paper towels. Add either 250 μL/well of HBSS+5% FCCS to the plank (no primary antibody wells) or 250 μL/well of primary antibody diluted in HBSS+5% FCS. Incubate for 30 minutes at 37° C. Wash the wells twice with 0.5 mL/well HBSS or PBS and gently tap the plates onto paper towels after the last wash. Add 250 μL/well of HRP-conjugated second antibody diluted in HBSS+5% FCS to every well including the blank wells (no primary antibody). Incubate at 37° C. for 30 minutes. Wash the wells four times with 0.5 mL/well HBSS or PBS and gently tap the plates onto paper towels after the last wash. Add 250 μL/well of substrate solution. Incubate at room temperature in the dark until there is adequate color development (blue). Note the length of time incubation was performed (typically 15-30 minutes). Add 75 μL/well stopper solution (8N sulfuric acid), and read at 450 nm.

The degree of inhibition of the compounds of formulas (I), (II) and (III) was determined by the assays described in Examples 5-6. The results are provided in Table I.

TABLE I

| | VCAM-1 IC₅₀ or | |
|---|---|---|
| Compound | inhibition at [μM] | LD₅₀ |
| 2,6-di-tert-butyl-4- | 80 | 200 |
| thio-(4′(methyl)phenyl | | |
| acetic acid)) phenol | | |
| 2,6-di-tert-butyl-4- | 10 | 200 |
| thio(4′-nitrobenzyl) | | |
| phenol | | |
| 2,6-di-tert-butyl-4- | 15 | 0.4 |
| thio(4′-nitrophenethyl) | | |
| phenol | | |
| 2,6-di-tert-butyl-4- | 75 | 200 |
| thio(butanoic | | |
| acid)phenol | | |
| 2,6-di-tert-butyl-4- | 6 | 50 |
| thio(3′,5′-ditert- | | |
| butyl,4′- | | |
| hydroxy butanedioic | | |
| acid ester)phenol | | |
| 2,6-di-tert-butyl-4- | NE | >100 |
| thio(4′(methyl)benzoic | | |
| acid)phenol | | |
| 2,6-di-tert-butyl-4- | 50 | |
| thio(2′-acetoxy,2′- | | |
| methylpropyl)phenol | | |
| 2,6-di-tert-butyl-4- | 13 | 200 |
| thio(3′- | | |
| nitrobenzyl)phenol | | |
| 2,6-di-tert-butyl-4- | 8 | 400 |
| thio(2′,4′- | | |
| dinitrobenzyl)phenol | | |
| (2,6-di-tert-butyl-4- | 5 | 300 |
| thio(4′- | | |
| (trifluoromethyl) | | |
| benzyl) phenol | | |
| 2,6-di-tert-butyl-4- | 40 | 400 |
| thio((2′-furancarboxylic | | |
| acid)-5-methyl)phenol | | |
| 2,6-di-tert-butyl-4- | 20 | 350 |
| thio(4′-methyl-N,N- | | |
| dimethylbenzene- | | |
| sulfonamide)phenol | | |
| 2,6-di-tert-butyl-4- | 50 | <100 |
| sulfinyl(4′- | | |
| nitrobenzyl)phenol | | |
| 2,6-di-tert-butyl-4- | 40 | 100 |
| (sulfonyl-(4′- | | |
| nitrobenzyl))phenol | | |
| 2,6-di-tert-butyl-4- | 18 | 75 |
| thio(4′-acetoxybenzyl) | | |
| phenol | | |
| 2,6-di-tert-butyl-4- | 75 | |
| thio(4′-methylbenzyl) | | |
| phenol | | |
| 2,6-di-tert-butyl-4- | 35 | |
| thio(4′-fluorobenzyl) | | |
| phenol | | |
| 2,6-di-tert-butyl-4- | 25% at 50 | |
| thio(3′-propanesulfonic | | |
| acid)phenol | | |
| 2,6-di-tert-butyl-4- | 10 | |
| thio(5′-methyl-2′- | | |
| ((dimethylamino)methyl) | | |
| furan)phenol | | |
| 2,6-di-tert-butyl-4- | 30% at 50 | 100 |
| thio(3′- | | |
| (dimethylamino)propyl)) | | |
| phenol | | |
| 2,6-di-tert-butyl-4- | 40% at 50 | 100 |
| thio((1′- | | |
| (acetoxy))pentyl)phenol | | |
| 2,6-di-tert-butyl-1- | NE | |
| methoxy-4-thio(4′- | | |
| trifluoromethyl)benzyl) | | |
| benzene | | |
| 2,6-di-tert-butyl-4- | 15 | 50 |
| thio(4′- | | |
| (methyl)phenylethyl | | |
| alcohol))phenol | | |
| Phenol, 4-[[1-[3,5- | 30% at 50 | >100 |
| bis(1,1-dimethylethyl)4- | | |
| [(4-nitrophenyl) | | |
| methoxy]phenyl] | | |
| thio]-1-methylethyl] | | |
| thio]2,6-bis(1,1- | | |
| dimethylethyl)- | | |
| Butanedioic acid, mono | 5.6 | 23 |
| [4-[[1-[[3,5-bis(1,1- | | |
| dimethylethyl)-4- | | |
| hydroxyphenyl]thio]-1- | | |
| methylethyl]thio]2,6- | | |
| bis(1,1-dimethylethyl) | | |
| phenyl] ester | | |
| 2-Furancarboxylic acid, | 25 | 400 |
| 5-nitro-, 4-[[1-[[3,5- | | |
| bis(1,1-dimethylethyl)- | | |
| 4-hydroxyphenyl]thio]- | | |
| 1-methylethyl]thio]-2,6- | | |
| bis(1,1-dimethylethyl) | | |
| phenyl ester | | |
| Butanoic acid, 4-[4-[[1- | 19 | 75 |
| [[(3,5-bis(1,1- | | |
| dimethylethyl)-4- | | |
| hydroxyphenyl]thio]-1- | | |
| methylethyl]thio]2,6- | | |
| dimethylphenoxy]- | | |
| Phenol, 4-[[1-[[4-(4- | 8 | 25 |
| aminobutoxy)-3,5- | | |
| bis(1,1-dimethylethyl) | | |
| phenyl]thio]-1- | | |
| methylethyl]thio]2,6- | | |
| bis(1,1-dimethylethyl)- | | |
| Phenol, 4-[[1-[[4-(4- | 9 | 25 |
| aminobutoxy)-3,5- | | |
| bis(1,1-dimethylethyl) | | |
| phenyl]thio]-1- | | |
| methylethyl]thio]2,6- | | |
| bis(1,1-dimethylethyl)- | | |
| Butanoic acid, 4- | 6 | 250 |
| hydroxy-, 4-[[1-[[3,5- | | |
| bis(1,1-dimethylethyl)- | | |
| 4-hydroxyphenyl]thio]- | | |
| 1-methylethyl]thio]-2,6- | | |
| bis(1,1-dimethylethyl) | | |
| phenyl ester | | |
| Propanoic acid, 2,2- | 25% at 25 | |
| dimethyl-, [4-[[1-[[3,5- | | |
| bis(1,1-dimethylethyl)- | | |
| 4-hydroxyphenyl]thio]-1- | | |
| methylethyl]thio]-2,6- | | |
| bis(1,1-dimethylethyl) | | |
| phenoxy] methyl ester | | |
| Phenol, 4-[[1-[[4-(4- | 5 | 12.5 |
| aminobutoxy)phenyl] | | |
| thio]-1- | | |
| methylethyl]thio]- | | |
| 2,6-bis(1,1- | | |
| dimethylethyl)- | | |
| Butanoic acid, 4-[4-[[1- | 19 | >100 |
| [[3,5-bis(1,1- | | |
| dimethylethyl)-4- | | |
| hydroxyphenyl]thio]-1- | | |
| methylethyl]thio] | | |
| phenoxy]- | | |
| Acetic acid, [4-[[1- | 10 | 50 |
| [[3,5- | | |
| bis(1,1-dimethylethyl)- | | |
| 4-hydroxyphenyl]thio]- | | |
| 1-methylethyl]thio]2,6- | | |
| bis(1,1-dimethylethyl) | | |
| phenoxy]- | | |
| Butanoic acid, 4-amino- | 8 | 25 |
| 4-oxo-, 4-[[1-[[3,5- | | |
| bis(1,1-dimethylethyl)- | | |
| 4-hydroxyphenyl]thio]- | | |
| 1-methylethyl]thio]-2,6- | | |
| bis(1,1-dimethylethyl) | | |
| phenyl ester | | |
| Glycine, 4-[[1-[[3,5- | 10% at 20 | 35 |
| bis(1,1-dimethylethyl)- | | |
| 4-hydroxyphenyl]thio]- | | |
| 1-methylethyl]thio]-2,6- | | |
| dimethylphenyl ester | | |
| Butanedioic acid, | 8 | 20 |
| mono[4-[[1-[[3,5- | | |
| bis(1,1-dimethylethyl)- | | |
| 4-hydroxyphenyl]thio)- | | |
| 1-methylethyl}-2,6- | | |
| dimethylphenyl]ester | | |
| Butanedioic acid, | 40% at 100 | |
| mono[4-[[1-[[3,5- | | |
| bis(1,1-dimethylethyl)- | | |
| 4-hydroxyphenyl]thio)- | | |
| 1-methylethyl}thio-2,6- | | |
| bis(1,1-dimethylethyl) | | |
| phenyl methyl ester | | |
| Glycine, 4-[[1-[[3,5- | 5 | 25 |
| bis(1,1-dimethylethyl)- | | |
| 4-hydroxyphenyl]thio]-1- | | |
| methylethyl]thio]-2,6- | | |
| bis(1,1-dimethylethyl) | | |
| phenyl ester | | |
| Pentanedioic acid, (1- | NE | 25 |
| methylethylidene)bis | | |
| (thio{2,6-bis(1,1- | | |
| dimethylethyl)-4,1- | | |
| phenylene)]ester | | |
| Pentanedioic acid | 8.7 | 25 |
| mono[4-[[1-[[3,5- | | |
| bis(1,1-dimethylethyl)- | | |
| 4-hydroxyphenyl]thio]-1- | | |
| methylethyl]thio]-2,6- | | |
| bis(1,1-dimethylethyl) | | |
| phenyl] ester | | |
| Butanoic acid, 4-[4-[[1- | 11 | 25 |
| [[3,5-bis(1,1- | | |
| dimethylethyl)-4- | | |
| hydroxyphenyl]thio]-1- | | |
| methylethyl]thio]-2,6- | | |
| bis(1,1- | | |
| dimethylethyl)phenoxy]- | | |
| Butanedioic acid, (1- | NE | 25 |
| methylethylidine)bis | | |
| [thio[2,6-bi(1,1- | | |
| dimethylethyl)-4,1- | | |
| phenylene}}ester, | | |
| Glycine, (1- | NE | |
| methylethylidene)bis | | |
| [bis[thio-2,6-bis(1,1- | | |
| dimethylthyl)-4,1- | | |
| phenylene]]ester, | | |
| dihydrochloride | | |
| Oxiranemethanol, α-[[4- | 45 | |
| [[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenoxy] methyl]-; | | |
| Oxiranemethanol, 3-[[4- | >100 | |
| [[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenoxy] methyl]-; | | |
| Oxiranemethanol, α- | 60 | |
| [[[3-[[4-[[1-[[3,5- | | |
| bis(1,1-dimethylethyl)- | | |
| 4-hydroxyphenyl]thio]-1- | | |
| methylethyl]thio]-2,6- | | |
| bis(1,1-dimethylethyl) | | |
| phenoxy]methyl]oxiranyl] | | |
| methoxy]methyl]- | | |
| Phenol, 4-[[1-[[3,5- | NE at 50 | |
| bis(1,1-dimethylethyl)- | | |
| 4-(oxiranylmethoxy) | | |
| phenyl]thio]-1- | | |
| methylethyl]thio]-2,6 | | |
| bis(1,1-dimethylethyl)- | | |
| Glycine, N-[3-[4-[[1- | 16 | 50 |
| [[3,5-bis(1,1- | | |
| dimethylethyl)-4- | | |
| hydroxyphenyl]thio]-1- | | |
| methylethyl]thio]-2,6- | | |
| bis(1,1- | | |
| dimethylethyl)phenoxy] | | |
| 2-hydroxypropyl]- | | |
| 1,2,3-Butanetriol, 4-[4- | 6 | 20 |
| [[1-[[3,5-bis(1,1- | | |
| dimethylethyl)-4- | | |
| hydroxyphenyl]thio]-1- | | |
| methylethyl]thio]-2,6- | | |
| bis(1,1- | | |
| dimethylethyl)phenoxy]- | | |
| 2-hydroxypropyl]- | | |
| Phenol, 4-[[1-[[3,5- | 75 | |
| bis(1,1-dimethylethyl)- | | |
| 4-(3-ethoxy-2- | | |
| hydroxypropoxy)phenyl] | | |
| thio]-1- | | |
| methylethyl]thio]-2,6- | | |
| bis(1,1-dimethylethyl)-; | | |
| 1,2-Propanediol, 3-[4- | 30 | |
| [[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenoxy]- | | |
| Phenol, 4-[[1-[[3,5- | NE at 50 | |
| bis(1,1-dimethylethyl)- | | |
| 4-ethoxyphenyl]thio]-1- | | |
| methylethyl]thio]-2,6- | | |
| bis(1,1-dimethylethyl)- | | |
| 2-Propanoic acid, 3-[4- | NE at 50 | |
| [[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenoxy]-,ethyl ester, | | |
| (E)- | | |
| Butanedioic acid, | NE | |
| mono[4-[[1-[[3,5- | | |
| bis(1,1-dimethylethyl)- | | |
| 4-methoxyphenyl]thio]1- | | |
| methylethyl]thio]-2,6- | | |
| bis(1,1-dimethylethyl) | | |
| phenyl] ester | | |
| Phenol, 4-[[1-[[4-[2-[4- | 55 | |
| (dimethylamino)phenyl] | | |
| ethoxy]-3,5-bis(1,1- | | |
| dimethylethyl)phenyl] | | |
| thio]-1- | | |
| methylethyl]thio]- | | |
| 2,6-bis(1,1- | | |
| dimethylethyl)- | | |
| Benzenamine, 4,4′- | NE | |
| [(1-methylethylidene) | | |
| bis [thio[2,6-bis(1,1- | | |
| dimethylethyl)-4,1- | | |
| phenylene]oxy-2,1- | | |
| ethanediyl]]bis[N,N- | | |
| dimethyl- | | |
| L-Arginine, mono[4-[[1- | 15 | 50 |
| [[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenyl butanedioate] | | |
| pentanedioic acid, 4- | 80 | |
| [[1-[[3,5-bis(1,1- | | |
| dimethylethyl)-4- | | |
| hydroxyphenyl]thio]-1- | | |
| methylethyl]thio]-,6- | | |
| bis(1,1-dimethylethyl) | | |
| phenylmethyl ester | | |
| 2-Propenoic acid, 3-[4- | 30 | |
| [[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenoxy]-, (E)- | | |
| α-D-Galactopyranose, 6- | 45 | |
| O-[4-[[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenyl]-1,2:3,4-bis-O- | | |
| (1-methylethylidene) | | |
| Phenol, 4-[[1-[[4-[3- | 22% at 50 | |
| (dimethylamino) | | |
| propoxy]-3,5-bis(1,1- | | |
| dimethylethyl)phenyl] | | |
| thio]-1- | | |
| methylethyl]thio]- | | |
| 2,6-bis(1,1- | | |
| dimethylethyl)- | | |
| Glycine, N-[[4-[[1- | 15 | 50 |
| [[3,5-bis(1,1- | | |
| dimethylethyl)-4- | | |
| hydroxyphenyl]thio]- | | |
| 1,methylethyl]thio]-2,6- | | |
| bis(1,1-dimethylethyl) | | |
| phenoxy]acetyl]- | | |
| Glutamic acid, N-[[4- | 75 | 100 |
| [[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenoxy]acetyl]- | | |
| L-Glutamic acid, N-[3- | 10 | 50 |
| [4-[[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenoxy]- | | |
| 2-hydroxypropyl]-di-, | | |
| diethyl ester | | |
| Glycine, N-[4-[4[[1- | 50 | >100 |
| [[3,5-bis(1,1- | | |
| dimethylethyl)-4- | | |
| hydroxphenyl]thio]-1- | | |
| methylethyl]thio]-2,6- | | |
| bis(1,1-dimethylethyl) | | |
| phenoxy]-2,3- | | |
| dihydroxybutyl]- | | |
| L-Lysine, N.sup.6-[3- | 75 | 100 |
| [4-[[1-[[3,5-bis(1,1- | | |
| dimethylethyl)-4- | | |
| hydroxyphenyl]thio]-1- | | |
| methylethyl]thio]- | | |
| 2,6-bis(1,1- | | |
| dimethylethyl)phenoxy]- | | |
| 2-hydroxypropyl]- | | |
| 2-Propenoic acid, 4-[4- | 75 | |
| [[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenoxy]butyl ester | | |
| Phenol, 4-[[1-[[3,5- | 125 | |
| bis(1,1-dimethylethyl)- | | |
| 4-(4-hydroxybutoxy) | | |
| phenyl]thio]-1- | | |
| methylethyl]thio]-2,6- | | |
| bis(1,1-dimethylethyl)- | | |
| β-D-Glucopyranose, 6- | 30% at 50 | |
| O-[4-[[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenyl]- | | |
| 1-H-Tetrazole-1- | 25% at 50 | |
| butanoic acid, 4-[[1- | | |
| [[3,5-bis(1,1 | | |
| dimethylethyl)-4- | | |
| hydroxyphenyl]thio]-1- | | |
| methylethyl]thio]-2,6- | | |
| bis(1,1-dimethylethyl) | | |
| phenyl ester | | |
| Phenol, 4-[[1-[[3,5- | 55 | |
| bis(1,1-dimethylethyl)- | | |
| 4-[[3-hydroxy-1- | | |
| propenyl)oxy]phenyl] | | |
| thio]-1- | | |
| methylethyl]thio]- | | |
| 2,6-bis(1,1- | | |
| dimethylethyl)- | | |
| L-Lysine, N⁶-[[4-[[1- | 30% at 50 | NE |
| [3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenoxy]acetyl]- | | |
| D-Glucopyranose, 6-O- | 10 | 50 |
| [4-[[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenyl]- | | |
| D-Glucitol, 6-O-[4-[[1- | 15 | 50 |
| [[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenyl]- | | |
| Butanoic acid, 4- | 43 | 75 |
| [[hydroxy(2- | | |
| hydroxyphenoxy) | | |
| phosphinyl]oxy]-4-[[1- | | |
| [[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenyl ester | | |
| Butanoic acid, 4- | 110 | |
| hydroxy-3,3-dimethyl-, | | |
| 4-[[1-[[3,5-bis(1,1- | | |
| dimethylethyl)-4- | | |
| hydroxyphenyl]thio]-1- | | |
| methylethyl]thio]-2,6- | | |
| bis(1,1-dimethylethyl) | | |
| phenyl ester | | |
| Butanoic acid, 4- | 20 | 50 |
| (sulfoxy)-, 1-[4-[[3,5- | | |
| bis(1,1-dimethylethyl)- | | |
| 4-hydroxyphenyl]thio]- | | |
| 1-methylethyl]thio]-2,6- | | |
| bis(1,1-dimethylethyl) | | |
| phenyl] ester | | |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept and scope of the invention.

What is claimed is:

1. A method for treating diabetic vascular disease, diabetic neuropathy, nephropathy, or retinopathy in a mammal, the method comprising administering to the mammal an effective amount of a compound of formula I 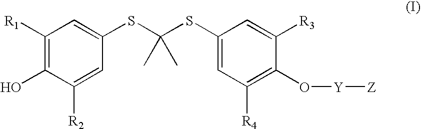
or a pharmaceutically acceptable salt thereof, wherein:
Y is a bond or 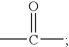
R₁, R₂, R₃ and R₄ are independently selected from the group consisting of hydrogen, hydroxy, alkyl, aryl, heteroaryl, alkaryl, and arylalkyl, wherein all nonhydrogen and hydroxy substituents may optionally be substituted from one or more of the group selected from alkyl, halogen, nitro, amino, haloalkyl, alkylamino, dialkylamino, acyl, and acyloxy;
Z is selected from the group consisting of alkyl, alkenyl, alkynyl, hydroxyl alkyl, aryl, heteroaryl, alkaryl, arylalkyl, heteroarylalkyl, alkoxy alkyl, alkylamino alkyl, carboxy alkyl, dialkylamino alkyl, amino alkyl, heterocycle, heterocycl alkyl, R₇NH, R₇ R₇N, carboxy, carbohydrate group, carbohydrate lactone group, and an alditol group wherein all may optionally be substituted by one or more R₅;
R₅ is independently selected from the group selected from the group selected from hydroxy, alkyl, alkoxy, halo, nitro, amino, cyano, alkylamino, dialkylamino, acyl, acyloxy, COOH, COOR₇, OC(O)R₇, CH(OH)R₇, NHR₇, NR₇ R₇, C(O)NH₂, C(O)NHR₇, CONR₇ R₇, NHC(O)O—R₇, OSO₃ H, SO₃ H, SO₂ NHR7, SO₂ NR₇ R₇, P(O)(OH)OR₇, PO₂H₂P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, alkyl, alkoxy, acyloxy, halo, nitro, amino, cyano, halo alkyl, alkylamino, di alkylamino, acyl, and acyloxy;
R₇ is independently selected from the group consisting of alkyl, alkenyl, alkynyl, alkoxy, alkoxycarbonyl alkyl, aryl, carboxy alkyl, alkylcarboxy alkyl, alkylcarboxy aryl, heterocycle, heterocycl alkyl, and heteroaryl, wherein all may be optionally substituted by one or more R₈; and
R₈ is independently selected from the group consisting of hydroxy, alkyl, alkoxy, acyloxy, halo, nitro, amino, cyano, and carboxy;
wherein two R₇ groups may come together to form a 4 to 7 membered ring.

2. A method for treating diabetic vascular disease in a mammal, the method comprising administering to the mammal an effective amount of a compound of formula I 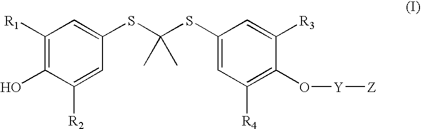
or a pharmaceutically acceptable salt thereof, wherein:
Y is a bond or 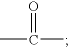
R₁, R₂, R₃ and R₄ are independently selected from the group consisting of hydrogen, hydroxy, C₁₋₁₀ alkyl, aryl, heteroaryl, C₁₋₁₀ alkaryl, and aryl C₁₋₁₀ alkyl, wherein all nonhydrogen and hydroxy substituents may optionally be substituted from one or more of the group selected from C₁₋₁₀ alkyl, halogen, nitro, amino, haloC₁₋₁₀ alkyl, C₁₋₁₀ alkylamino, diC₁₋₁₀ alkylamino, acyl, and acyloxy;
Z is selected from the group consisting of C₁₋₁₀ alkyl, C₂₋₁₀ alkenyl, C₂₋₁₀ alkynyl, hydroxyl C₁₋₁₀ alkyl, aryl, heteroaryl, C₁₋₁₀ alkaryl, aryl C₁₋₁₀ alkyl, heteroaryl C₁₋₁₀ alkyl, C₁₋₁₀ alkoxy C₁₋₁₀ alkyl, C₁₋₁₀ alkylamino C₁₋₁₀ alkyl, carboxy C₁₋₁₀ alkyl, C₁₋₁₀ dialkylamino C₁₋₁₀ alkyl, amino C₁₋₁₀ alkyl, heterocycle, heterocycl C₁₋₁₀ alkyl, R₇NH, R₇ R₇N, carboxy, carbohydrate group, carbohydrate lactone group, and an alditol group wherein all may optionally be substituted by one or more R₅;
R₅ is independently selected from the group selected from the group selected from hydroxy, C₁₋₁₀ alkyl, C₁₋₁₀ alkoxy, halo, nitro, amino, cyano, C₁₋₁₀ alkylamino, diC₁₋₁₀ alkylamino, acyl, acyloxy, COOH, COOR₇, OC(O)R₇, CH(OH)R₇, NHR₇, NR₇ R₇, C(O)NH₂, C(O)NHR₇, CONR₇ R₇, NHC(O)O—R₇, OSO₃ H, SO₃ H, SO₂ NHR7, SO₂ NR₇ R₇, P(O)(OH)OR₇, PO₂H₂P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₁₀ alkyl, C₁₋₁₀ alkoxy, acyloxy, halo, nitro, amino, cyano, haloC₁₋₁₀ alkyl, C₁₋₁₀ alkylamino, diC₁₋₁₀ alkylamino, acyl, and acyloxy;
R₇ is independently selected from the group consisting of C₁₋₁₀ alkyl, C₂₋₁₀ alkenyl, C₂₋₁₀ alkynyl, C₁₋₁₀ alkoxy, C₁₋₁₀ alkoxycarbonyl C₁₋₁₀ alkyl, aryl, carboxy C₁₋₁₀ alkyl, C₁₋₁₀ alkylcarboxy C₁₋₁₀ alkyl, C₁₋₁₀ alkylcarboxy C₁₋₁₀ aryl, heterocycle, heterocycl C₁₋₁₀ alkyl, and heteroaryl, wherein all may be optionally substituted by one or more R₈; and
R₈ is independently selected from the group consisting of hydroxy, C₁₋₁₀ alkyl, C₁₋₁₀ alkoxy, acyloxy, halo, nitro, amino, cyano, and carboxy;
wherein two R₇ groups may come together to form a 4 to 7 membered ring.

3. A method for treating diabetic neuropathy, nephropathy, or retinopathy in a mammal, the method comprising administering to the mammal an effective amount of a compound of formula I 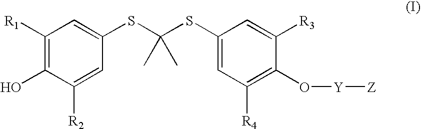
or a pharmaceutically acceptable salt thereof, wherein:
Y is a bond or 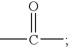
R₁, R₂, R₃ and R₄ are independently selected from the group consisting of hydrogen, hydroxy, C₁₋₁₀ alkyl, aryl, heteroaryl, C₁₋₁₀ alkaryl, and aryl C₁₋₁₀ alkyl, wherein all nonhydrogen and hydroxy substituents may optionally be substituted from one or more of the group selected from C₁₋₁₀ alkyl, halogen, nitro, amino, haloC₁₋₁₀ alkyl, C₁₋₁₀ alkylamino, diC₁₋₁₀ alkylamino, acyl, and acyloxy;
Z is selected from the group consisting of C₁₋₁₀ alkyl, C₂₋₁₀ alkenyl, C₂₋₁₀ alkynyl, hydroxyl C₁₋₁₀ alkyl, aryl, heteroaryl, C₁₋₁₀ alkaryl, aryl C₁₋₁₀ alkyl, heteroaryl C₁₋₁₀ alkyl, C₁₋₁₀ alkoxy C₁₋₁₀ alkyl, C₁₋₁₀ alkylamino C₁₋₁₀ alkyl, carboxy C₁₋₁₀ alkyl, C₁₋₁₀ dialkylamino C₁₋₁₀ alkyl, amino C₁₋₁₀ alkyl, heterocycle, heterocycl C₁₋₁₀ alkyl, R₇NH, R₇ R₇N, carboxy, carbohydrate group, carbohydrate lactone group, and an alditol group wherein all may optionally be substituted by one or more R₅;
R₅ is independently selected from the group selected from the group selected from hydroxy, C₁₋₁₀ alkyl, C₁₋₁₀ alkoxy, halo, nitro, amino, cyano, C₁₋₁₀ alkylamino, diC₁₋₁₀ alkylamino, acyl, acyloxy, COOH, COOR₇, OC(O)R₇, CH(OH)R₇, NHR₇, NR₇ R₇, C(O)NH₂, C(O)NHR₇, CONR₇ R₇, NHC(O)O—R₇, OSO₃ H, SO₃ H, SO₂ NHR7, SO₂ NR₇ R₇, P(O)(OH)OR₇, PO₂H₂P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₁₀ alkyl, C₁₋₁₀ alkoxy, acyloxy, halo, nitro, amino, cyano, haloC₁₋₁₀ alkyl, C₁₋₁₀ alkylamino, diC₁₋₁₀ alkylamino, acyl, and acyloxy;
R₇ is independently selected from the group consisting of C₁₋₁₀ alkyl, C₂₋₁₀ alkenyl, C₂₋₁₀ alkynyl, C₁₋₁₀ alkoxy, C₁₋₁₀ alkoxycarbonyl C₁₋₁₀ alkyl, aryl, carboxy C₁₋₁₀ alkyl, C₁₋₁₀ alkylcarboxy C₁₋₁₀ alkyl, C₁₋₁₀ alkylcarboxy C₁₋₁₀ aryl, heterocycle, heterocycl C₁₋₁₀ alkyl, and heteroaryl, wherein all may be optionally substituted by one or more R₈; and
R₈ is independently selected from the group consisting of hydroxy, C₁₋₁₀ alkyl, C₁₋₁₀ alkoxy, acyloxy, halo, nitro, amino, cyano, and carboxy;
wherein two R₇ groups may come together to form a 4 to 7 membered ring.

4. A method for treating diabetic vascular disease in a mammal, the method comprising administering to the mammal in need thereof an effective amount of a compound of Formula II 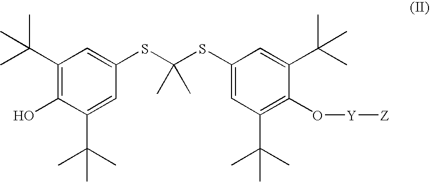
or a pharmaceutically acceptable salt thereof, wherein:
Y is a bond or 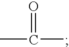
Z is selected from the group consisting of C₁₋₁₀ alkyl, C₂₋₁₀ alkenyl, C₂₋₁₀ alkynyl, hydroxyl C₁₋₁₀ alkyl, aryl, heteroaryl, C₁₋₁₀ alkaryl, aryl C₁₋₁₀ alkyl, heteroaryl C₁₋₁₀ alkyl, C₁₋₁₀ alkoxy C₁₋₁₀ alkyl, C₁₋₁₀ alkylamino C₁₋₁₀ alkyl, carboxy C₁₋₁₀ alkyl, C₁₋₁₀ dialkylamino C₁₋₁₀ alkyl, amino C₁₋₁₀ alkyl, heterocycle, heterocycl C₁₋₁₀ alkyl, R₇NH, R₇ R₇N, carboxy, carbohydrate group, carbohydrate lactone group, and an alditol group wherein all may optionally be substituted by one or more R₅;
R₅ is independently selected from the group selected from the group selected from hydroxy, C₁₋₁₀ alkyl, C₁₋₁₀ alkoxy, halo, nitro, amino, cyano, C₁₋₁₀ alkylamino, diC₁₋₁₀ alkylamino, acyl, acyloxy, COOH, COOR₇, OC(O)R₇, CH(OH)R₇, NHR₇, NR₇ R₇, C(O)NH₂, C(O)NHR₇, CONR₇ R₇, NHC(O)O—R₇, OSO₃ H, SO₃ H, SO₂ NHR7, SO₂ NR₇ R₇, P(O)(OH)OR₇, PO₂H₂P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₁₀ alkyl, C₁₋₁₀ alkoxy, acyloxy, halo, nitro, amino, cyano, haloC₁₋₁₀ alkyl, C₁₋₁₀ alkylamino, diC₁₋₁₀ alkylamino, acyl, and acyloxy;
R₇ is independently selected from the group consisting of C₁₋₁₀ alkyl, C₂₋₁₀ alkenyl, C₂₋₁₀ alkynyl, C₁₋₁₀ alkoxy, C₁₋₁₀ alkoxycarbonyl C₁₋₁₀ alkyl, aryl, carboxy C₁₋₁₀ alkyl, C₁₋₁₀ alkylcarboxy C₁₋₁₀ alkyl, C₁₋₁₀ alkylcarboxy C₁₋₁₀ aryl, heterocycle, heterocycl C₁₋₁₀ alkyl, and heteroaryl, wherein all may be optionally substituted by one or more R₈; and
R is independently selected from the group consisting of hydroxy, C₁₋₁₀ alkyl, C₁₋₁₀ alkoxy, acyloxy, halo, nitro, amino, cyano, and carboxy;
wherein two R₇ groups may come together to form a 4 to 7 membered ring.

5. The method of claim 4, wherein Y is a bond or C 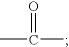
Z is selected from the group consisting of C₁₋₁₀ alkyl, C₂₋₁₀ alkenyl, C₂₋₁₀ alkynyl, hydroxyl C₁₋₁₀ alkyl, aryl, and heteroaryl, wherein all may optionally be substituted by one or more R₅;
R₅ is independently selected from the group consisting of hydroxyl, COOH, COOR₇, OC(O)R₇, CH(OH)R₇, NHR₇, NR₇R₇, C(O)NH₂, C(O)NHR₇, CONR₇R₇, NHC(O)O—R₇; and
R₇ is independently selected from the group consisting of C₁₋₁₀ alkyl, C₂₋₁₀ alkenyl, C₂₋₁₀ alkynyl, C₁₋₁₀ alkoxy, C₁₋₁₀ alkoxycarbonyl C₁₋₁₀ alkyl, aryl, carboxy C₁₋₁₀ alkyl, C₁₋₁₀ alkylcarboxy C₁₋₁₀ alkyl, C₁₋₁₀ alkylcarboxy C₁₋₁₀ aryl, heterocycle, heterocycl C₁₋₁₀ alkyl, and heteroaryl;
wherein two R₇ groups may come together to form a 4 to 7 membered ring.

6. The method of claim 4, wherein
Y is a bond or 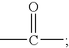
Z is selected from the group consisting of C₁₋₁₀ alkyl, C₂₋₁₀ alkenyl, C₂₋₁₀ alkynyl, hydroxyl C₁₋₁₀ alkyl, and aryl, wherein all may optionally be substituted by one or more R₅;
R₅ is independently selected from the group consisting of hydroxyl, COOH, COOR₇, OC(O)R₇; and
R₇ is independently selected from the group consisting of C₁₋₁₀ alkyl, C₂₋₁₀ alkenyl, C₂₋₁₀ alkynyl, and C₁₋₁₀ alkoxy.

7. The method of claim 4, wherein the compound or its pharmaceutically acceptable salt are selected from the group consisting of

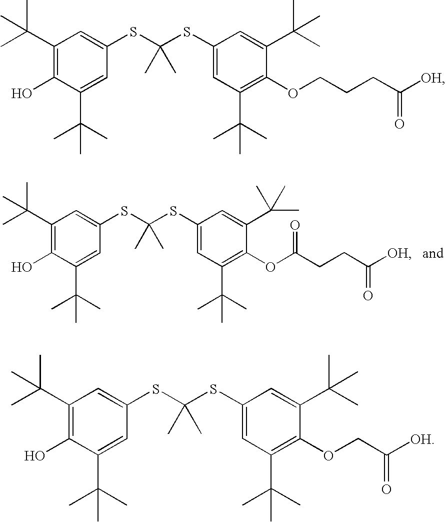

8. The method of claim 7, wherein the compound or its pharmaceutically acceptable salt is

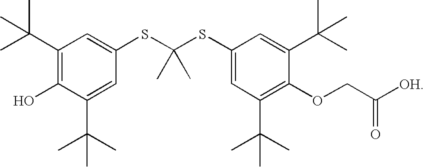

9. The method of claim 4, wherein the compound or its pharmaceutically acceptable salt are administered through a conjunctival administration, nasal administration, a buccal administration, an epidermal administration, or a parenteral administration.

10. The method of claim 4, wherein the compound or its pharmaceutically acceptable salt are administered orally.

11. The method of treating diabetic vascular disease according to claim 4, wherein the compound or its pharmaceutically acceptable salt are administered through a conjunctival administration.

12. The method of treating diabetic vascular disease according to claim 11, wherein the compound or its pharmaceutically acceptable salt is administered through the conjunctival administration in a form of an ophthalmic solution, an ophthalmic suspension, an ophthalmic gel, an ophthalmic ointment, or an ophthalmic strip or insert.

13. The method of claim 4, wherein the administration is in an amount of from about 0.01 mg/kg/day to about 1000 mg/kg/day.

14. The method of claim 4, wherein the therapeutically effective amount is in the form of a pharmaceutical formulation comprising the compound and a suitable carrier or excipient thereof.

15. A method for the treatment of a human afflicted with a diabetic vascular disease, the method comprising administering to the human in need of such a treatment a therapeutically effective amount of a composition comprising a compound represented by the formula: 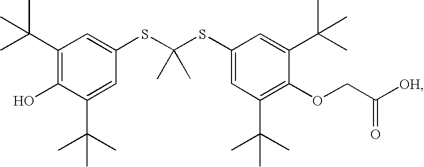
or a pharmaceutically acceptable salt thereof, optionally in a pharmaceutically acceptable carrier.
